(19) United States
(12) Patent Application Publication
(10) Pub. No.: US 2024/0130812 A1
(43) Pub. Date: Apr. 25, 2024

(54) SYSTEMS AND METHODS FOR CONTROL OF A SURGICAL SYSTEM

(71) Applicant: Intuitive Surgical Operations, Inc., Sunnyvale, CA (US)

(72) Inventor: Ashwinram SURESH, San Jose, CA (US)

(73) Assignee: Intuitive Surgical Operations, Inc., Sunnyvale, CA (US)

(21) Appl. No.: 18/377,959

(22) Filed: Oct. 9, 2023

Related U.S. Application Data
(60) Provisional application No. 63/416,149, filed on Oct. 14, 2022.

(51) Int. Cl. A61B 34/35; A61B 34/00; A61B 90/00

(52) U.S. Cl. CPC A61B 34/35; A61B 34/76; A61B 90/06; A61B 2034/305

(57) ABSTRACT

Systems and methods are provided for control of a surgical system. Accordingly, haptic feedback is provided to an input device of the surgical system based on a force applied to an instrument of the surgical system. The instrument includes a force sensor unit that has a beam, a first strain sensor coupled to the beam, and a second strain sensor. A first signal is received from the first strain sensor and is associated with a deflection of the beam in response to an applied force. A second signal is received from the second strain sensor and is associated with contact between the beam and a hard stop structure. The magnitude of the applied force is determined based at least on the first signal and the second signal.

### CROSS-REFERENCE TO RELATED APPLICATIONS

This patent application claims priority to and the filing date benefit of U.S. Provisional Patent Application No. 63/416,149, entitled “Systems and Methods for Control of a Surgical System” filed Oct. 14, 2022, the disclosure of which is incorporated herein by reference in its entirety.

### BACKGROUND

The embodiments described herein relate to surgical systems, and more specifically to teleoperated surgical systems. More particularly, the embodiments described herein relate to systems and methods for determining a deflection of a medical instrument in order to control a surgical system that includes a force feedback that may be provided to a system operator.

Known techniques for Minimally Invasive Surgery (MIS) employ instruments to manipulate tissue that can be either manually controlled or controlled via hand-held or mechanically grounded teleoperated medical systems that operate with at least partial computer-assistance (“telesurgical systems”). Many known MIS instruments include a therapeutic or diagnostic end effector (e.g., forceps, a cutting tool, or a cauterizing tool) mounted on an optional wrist mechanism at the distal end of a shaft. During an MIS procedure, the end effector, wrist mechanism, and the distal end of the shaft are typically inserted into a small incision or a natural orifice of a patient to position the end effector at a work site within the patient's body. The optional wrist mechanism can be used to change the end effector's position and orientation with reference to the shaft to perform a desired procedure at the work site. In known instruments, motion of the instrument as a whole provides mechanical degrees of freedom (DOFs) for movement of the end effector, and the wrist mechanisms generally provide the desired DOFs for movement of the end effector with reference to the shaft of the instrument. For example, for forceps or other grasping tools, known wrist mechanisms are able to change the pitch and yaw of the end effector with reference to the shaft. A wrist may optionally provide a roll DOF for the end effector, or the roll DOF can be implemented by rolling the shaft. An end effector may optionally have additional mechanical DOFs, such as grip or knife blade motion. In some instances, wrist and end effector mechanical DOFs and be combined. For example, U.S. Pat. No. 5,792,135 (filed May 16, 1997) discloses a mechanism in which wrist and end effector grip DOFs are combined.

Force sensing surgical instruments are known and together with associated telesurgical systems deliver haptic feedback to a surgeon performing an MIS procedure. The haptic feedback may increase the immersion, realism, and intuitiveness of the procedure for the surgeon. For effective haptics rendering and accuracy, force sensors can be placed on a medical instrument and as close to the anatomical tissue interaction as possible. One approach is to include a force sensor unit having electrical sensor elements (e.g., strain sensors or strain gauges) at a distal end of a medical instrument shaft to measure strain imparted to the medical instrument. The measured strain can be used to determine the force imparted to the medical instrument and as input from which the desired haptic feedback can be generated.

FIG. 1A shows one example of a known force sensor unit that includes a cantilever beam 810 attached between the instrument distal tip component 510 (e.g., in some cases a clevis or other wrist or end effector component) and the instrument shaft 410 that extends back to the mechanical structure. As illustrated, strain sensors 830 are coupled to the beam to measure strain in X- and Y-directions as shown(arbitrary Cartesian directions that are orthogonal to each other and to a longitudinal axis of the beam and instrument shaft). For example, the strain sensors can optionally include full Wheatstone bridges (full bridges). In some cases, in order to reject common modes, such as temperature, the strain sensors are each split into two sets, with one set on the distal end of the beam and the other set on the proximal end of the beam. Because the beam is secured to a distal portion of the instrument shaft, the strain sensors sense strain on the beam orthogonal to a longitudinal axis AA of the shaft. A force component F_{A} (FIG. 1B) applied orthogonal to the beam (i.e., a force in an X-Y plane, such as an X or Y force) is determined by subtracting strain measurements determined by the full-bridges at the proximal and distal end portions of that side face of the beam.

During the employment of the medical instrument, however, certain operating conditions may be encountered under which the output of the force sensor unit may not accurately indicate the force imparted to the medical instrument. The operating conditions may, for example, correspond to the positioning of the medical instrument, an operation being performed by the medical instrument, and/or a fault condition. The inaccuracies that may be encountered may limit the ability of the telesurgical system to deliver accurate haptic feedback to the surgeon performing the procedure.

For example, in certain positions, the strain indicated by the strain sensors may be less than the strain that would be imparted to the medical instrument in response to the applied force F_{A} affecting the distal tip component 510 when not in the certain positions. More specifically, some known force sensing medical instruments may include or be used with a substantially stiff structure 901 that at least partially surrounds the beam 810 and upon contact either stops beam 810's further deflection or effectively changes beam 810's stiffness and resulting deflection characteristics. For example, some known force sensing medical instruments may include a protective structure (e.g., shroud) that covers the strain sensors 830 and their associated wires during use. In other words, the structure 901 is a structure that does not deflect to the same degree as the beam 810. To ensure the beam 810 remains cantilevered for accurate force sensing, the structure 901 may not be directly coupled to the distal tip component 510. Instead, the structure 901 may be separate from the distal tip component to allow the beam to deflect when affected by the applied force F_{A} (see FIG. 1B). In certain situations, however, the distal end of the structure 901 may contact the beam (or a portion of the medical instrument surrounding the beam) or the distal tip component, thereby limiting deflection of the beam. FIG. 1B shows one example, in which the beam 810 is deflected in the X direction such that it contacts one side of the distal end of the structure 901 (e.g., the shroud), which limits or prevents further bending of the beam 810 in the X direction by an amount that is dependent upon structure 901's rigidity and the relative stiffeness between the structure 901 and the beam 810.

Although limiting the displacement of the beam can advantageously prevent overload of the beam 810 and/or the strain sensors 830, we have discovered that such known systems that engage the beam at a single point can cause a change in the strain distribution over the length of the beam 810. In other words, the beam 810 no longer functions as a cantilevered beam anchored solely at one end. The contact location between structure 901 and beam 810 acts as a fulcrum around which beam 810 bends. As a result, the strain sensors 830 produce signals that do not accurately represent the applied force F_{A} (e.g., the actual force affecting the medical instrument). Specifically, we have discovered that in certain situations the contact between the distal end of the structure 901 and the beam 810 may cause distortion of the signals produced by the strain sensors 830. In certain situations, the distortion can cause the force sensed by the strain sensors 830 to be in the opposite direction of the applied force F_{A} (this phenomenon can be referred to as “force inversion” because a human operator's haptic sensation of force direction based on the erroneous strain sensor signals will be inverted from the correct force direction).

FIGS. 2A and 2B are rigid body mechanics diagrams of example known force sensing medical instrument of FIGS. 1A and 1B to further illustrate this example of force distortion and inversion. As shown in FIG. 2A, the contact between the shroud and the beam can be modeled as a single point contact (at GND 2). In FIG. 2A, the distance L represents the distance from the base of the beam 810 (point GND 1) to the point where the shroud (e.g., the substantially stiff structure 901) contacts the beam 810 (point GND 2). The distance D represents the distance between the point where the shroud contacts the beam 810 (point GND 2) and location at which the applied force F_{A} is applied to or by the distal tip component 510.

FIG. 2B is a rigid body mechanics diagram of the beam showing exaggerated deflection of the beam as a result of the contact at point GND 2. As shown, we have discovered that the strain distribution along the top surface of the beam transitions from a proximal region of compression to a more distal region of tension, which causes the signals from the strain sensors 830 to inaccurately represent the applied force F_{A}.

FIG. 2C shows the modeled forces with the beam “cut” at point GND 2 for purposes of analyzing the force and pure moment of the beam. FIG. 2C shows the reactive force F_{R} produced by the single point contact, the consolidated force F (such as may be indicated by the strain sensors 830 of FIG. 1B), and a pure moment (M) (e.g., a couple) produced by the oposing force vectors of equal magnitude according to rigid body mechanics. By modeling the beam at the point of contact (at GND 2), the additional deflection (i.e., beyond this point of contact away from the shaft) can be considered as zero. Using the static and deflection equations shows that there are two different strain profiles over the entire beam length. The strain profile (ε) on the top side of the beam for the beam length l being between 0 and L is given by Eq. (1), where E is the modulus of elasticity of the beam, I is the moment of inertia of the XY cross section of the beam, and r is the perpendicular distance of the strain gauge from the neutral axis of the beam:

$$ε ( l ) = - \frac{F ( L - l ) r}{EI} + \frac{Mr}{EI} \qquad Eq . ( 1 )$$

The strain profile (ε) on the top side of the beam for the beam length l being between L and L+D is given by Eq. (2):

$$ε ( l ) = - \frac{{F}_{A} ( L + D - l ) r}{EI} \qquad Eq . ( 2 )$$

Thus, at certain locations along the beam 810, the strain sensors 830 produce signals indicative of the force F and not necessarily the applied force F_{A}. The signal associated with force F includes force components associated with both the applied force F_{A} and the reactive force F_{R}. This results in a distortion (and even an inversion of force direction) of the determined force relative to the applied force F_{A} that is actually exerted on the beam.

FIG. 3A is a graph showing the strain along the top of the beam 810 along the length of the beam based on Eq. (1) and Eq. (2) for the condition when the beam 810 substantially contacts the structure 901 at the single point of contact (GND 2). To further illustrate force distortion, FIG. 3B is a graph showing measured force (e.g., a determined force based on the signals) as a function of the actual force applied. As shown, when the beam 810 is not in contact with the shroud (e.g., the substantially stiff structure 901), for example, when the actual force applied does not cause sufficient bending of the beam 810 to result in the displacement of the beam 810 being affected by the shroud, the relationship between the measured force (e.g., force measurements derived from strain gauges) and the applied force (e.g., actual force component in the XY plane) is linear, which allows for an accurate calibration (i.e., based on the slope of the line). At conditions in which the beam 810 is in contact with the shroud (as shown in FIG. 1B and illustrated in FIG. 2B), however, the measured force decreases as the actual force increases.

When the measured force is used to produce haptic feedback to a person operating an instrument that includes the beam (e.g., haptic feedback at an input device the person is using to control the instrument), this measured force distortion/force inversion problem can result in an undesirable positive feedback loop, which could cause unexpected or undesirable movement at the input device. This discovery is more fully described in in U.S. Patent Publication No. US 2021/0353373 (filed May 17, 2021), entitled “Hard Stop that Produces a Reactive Moment Upon Engagement for Cantilever-Based Force Sensing,” which is incorporated herein by reference in its entirety for all purposes.

In view of this situation, the art is continuously seeking new and improved systems and methods for control of a surgical system.

### SUMMARY

This summary introduces certain aspects of the embodiments described herein to provide a basic understanding. This summary is not an extensive overview of the inventive subject matter.

The present disclosure includes systems and methods that facilitate the provision of haptic feedback to an input unit of a surgical system. The systems and methods disclosed herein use at least one sensor configured to detect contact between a beam of a force sensor unit and a surrounding hard stop structure that limits the deflection of the beam. The sensor(s) can also determine the magnitude of the force resulting from the contact between the beam and the hard stop structure. This magnitude, in conjunction with the output of a strain sensor coupled to the beam can be used to determine the magnitude of an applied force. The determined magnitude can then be used to generate haptic feedback that is delivered to an input device of a surgical system.

In one aspect, the present disclosure is directed to a force sensor unit, such as can be used with a medical instrument of a surgical system. The force sensor unit can be configured to measure an applied force, and haptic feedback can be generated based, at least in part, on the magnitude of the applied force. In some embodiments, the force sensor unit includes a beam that is at least partially surrounded by a hard stop structure. The beam includes a proximal end portion and a distal end portion. The hard stop structure can limit the deflection of the beam in response to an applied force. The hard stop structure includes a reference location at which the distal end portion of the beam contacts the hard stop structure on a condition in which a deflection of the beam is larger than a beam deflection threshold. A first strain sensor is coupled to the beam and configured to measure the applied force. Accordingly, the first strain sensor generates a first signal associated with the deflection of the beam. The force sensor unit also includes a second strain sensor. The second strain sensor produces a second signal in response to contact between the reference location and the distal end portion of the beam.

In some embodiments, the hard stop structure is a shroud or an outer shaft that surrounds at least a portion of the beam. The second strain sensor is positioned on the hard stop structure.

In some embodiments, the hard stop structure is the shroud. The shroud includes a wall surrounding the distal end portion of the beam. The shroud defines a hard stop engagement locator configured to engage the outer shaft or the beam in response to the deflection of the beam. The hard stop engagement locator defines a longitudinal position of the reference location. In some embodiments, the second strain sensor includes one or more strain gauges coupled to the shroud. The strain gauge(s) generates an output voltage indicative of a strain of the wall.

In some embodiments, the hard stop structure is the outer shaft. The outer shaft further includes a wall surrounding any of the beam and a portion of the shroud. The wall includes an inner face. The second strain sensor is coupled to the inner face of the wall at the reference location. In some embodiments, the second strain sensor includes at least one force sensor operably coupled to a load ring that is a deformable member. In some embodiments, the deformable member is an elastomeric member. The second strain sensor includes three or more force sensors embedded in the elastomeric member. In some embodiments, the force sensors detect force in two degrees of freedom. For example, in some embodiments, the force sensors are distributed equidistantly about a cross-sectional circumference of the outer shaft.

In some embodiments, the force sensor unit includes a contact feature positioned at a longitudinal location of the beam corresponding to the reference location and the second strain sensor is on the beam. The first strain sensor is positioned longitudinally between the reference location and the proximal end portion of the beam The second strain sensor is positioned longitudinally between the reference location and the distal end portion of the beam. In some embodiments, the force sensor unit is coupled between a surgical instrument shaft and a surgical instrument wrist assembly of a surgical instrument. The proximal end portion of the beam is coupled to the surgical instrument shaft and the distal end portion of the beam is coupled to the surgical instrument wrist assembly.

In one aspect, the present disclosure is directed to a surgical system. The surgical system includes a manipulator unit that supports and operates a medical instrument. The medical instrument includes a force sensor unit. The force sensor unit is configured to measure forces applied to the medical instrument. Accordingly, the force sensor unit includes a beam, a first strain sensor, and a second strain sensor. The beam includes a proximal end portion and a distal end portion. The first strain sensor is positioned to produce a first signal associated with a deflection of the beam. A hard stop structure at least partially surrounds the beam. The hard stop structure includes a reference location at which the distal end portion of the beam contacts the hard stop structure on a condition in which a deflection of the beam is larger than a beam deflection threshold. The surgical system also includes an input device and a controller. The controller operably couples the input device and the manipulator unit, and therefore, operably couples the force sensor unit in the input device. The controller translates operator inputs to the input device into movements and/or operations of the medical instrument. The controller includes a logic system and a memory system. The controller includes a haptic feedback module, and the controller is configured to perform a set of operations. The set of operations includes receiving a first signal from the first strain sensor. The first signal is associated with a strain associated with the deflection of the beam in response to an applied force. The set of operations include receiving a second signal from the second strain sensor. The second signal is produced in response to contact between the distal end portion of the beam and the reference location. The set of operations include determining, via the controller and based at least in part on the first signal and at least in part on the second signal, a determined magnitude of the applied force. Additionally, the set of operations includes, providing, via the haptic feedback module and based on the determined magnitude of the applied force, haptic feedback to a human operator of the input device.

In some embodiments, the second signal is indicative of whether the distal end portion of the beam is in contact with the hard stop structure at the reference location. On a condition in which the distal end portion of the beam is not in contact with the hard stop at the reference location, the haptic feedback is a designed haptic feedback. On a condition in which the distal end portion of the beam is in contact with the hard stop at the reference location, the haptic feedback is a modified haptic feedback. In some embodiments, the controller provides an indication to the operator of the input device that the modified haptic feedback is provided to, or is available to be provided to, the input device.

In some embodiments, the force sensor unit includes a contact feature positioned at a longitudinal location of the beam corresponding to the reference location, and the second strain sensor is on the beam. The first strain sensor is positioned longitudinally between the reference location and the proximal end portion of the beam, while the second strain sensor is positioned longitudinally between the reference location and the distal end portion of the beam. The second signal is indicative of the distal end portion of the beam being in contact with the hard stop at the reference location when the second signal deviates from a proximal strain function of the first signal. In some embodiments, the second signal corresponds to a reactive force magnitude that is larger than a reactive-force minimum threshold. When the reactive force magnitude is larger than the reactive-force maximal threshold, the set of operations include halting an operation of the surgical system. As such, the modified haptic feedback corresponds to the halted state of the surgical system.

In some embodiments, determining the magnitude of the applied force includes determining, via the controller, a strain magnitude at the reference location based on the proximal strain function. A distal strain function is determined by the controller based on the strain magnitude at the reference location and the second signal, and the determined magnitude of the applied force is proportional to a slope of the distal strain function.

In one aspect, the present disclosure is directed to a method of control for a surgical system. The surgical system includes a controller, an input device, and a medical instrument. The medical instrument is operably coupled to the input device via the controller. The controller translates operator inputs to the input device into movements and/or operations of the medical instrument. The medical instrument includes a force sensor unit. The force sensor unit includes a beam. A first strain sensor is coupled to the beam. The force sensor unit also includes a second strain sensor. The method can include, any of the operations disclosed herein.

These and other features, aspects and advantages of the present invention will become better understood with reference to the following description and drawings.

### BRIEF DESCRIPTION OF THE DRAWINGS

FIGS. 1A and 1B are diagrammatic illustrations of a portion of a known medical device including a force sensor unit in a first configuration (FIG. 1A) and a second configuration (FIG. 1B).

FIGS. 2A and 2B are rigid body mechanics diagrams of the portion of the medical device shown in FIGS. 1A and 1B in the first configuration (FIG. 2A) and showing an exaggerated beam displacement (FIG. 2B).

FIG. 2C is a rigid body mechanics diagram of the portion of the medical device shown in FIGS. 1A and 1B being analyzed at a point of contact.

FIG. 3A is a graph showing the surface strain along the length of a beam of a force sensor unit when a single point of contact occurs.

FIG. 3B is a graph showing the determined force (Y-axis) as a function of the actual force (X-axis) to demonstrate determined force distortion.

FIG. 4 is a diagrammatic plan view of a minimally invasive teleoperated medical system according to an embodiment being used to perform a medical procedure, such as surgery.

FIG. 5 is a diagrammatic plan view of the minimally invasive teleoperated medical system of FIG. 4 being used to perform a medical procedure, such as surgery.

FIG. 6 is a front perspective view of a user control console of the minimally invasive teleoperated surgery system shown in FIG. 5, according to an embodiment.

FIG. 7 is a perspective view of an input device of the user console shown in FIG. 6.

FIG. 8 illustrates a displayed view of a surgical site as presented to an operator of the minimally invasive teleoperated surgery system by the user control console shown in FIG. 6.

FIG. 9 is a front perspective view of an optional auxiliary unit of the minimally invasive teleoperated surgery system shown in FIG. 5.

FIG. 10 is a side elevation view of a manipulator unit, including a plurality of manipulators and instruments, of the minimally invasive teleoperated surgery system shown in FIG. 5.

FIG. 11 is a diagrammatic illustration of a portion of a medical instrument including a force sensor unit in a neutral orientation.

FIG. 12 is an enlarged view of a portion of the medical instrument of FIG. 11 indicated by the region K₁.

FIG. 13 is a diagrammatic illustration of a portion of a medical instrument of FIG. 11 including a force sensor unit in a deflected orientation.

FIG. 14 is an enlarged view of a portion of the medical instrument of FIG. 13 in the deflected orientation indicated by the region K₂.

FIG. 15 is a graph showing the determined force (Y-axis) as a function of the actual force (X-axis) during operation of the medical instrument of FIG. 11 on a condition in which the beam deflection is less than a deflection threshold and on a condition in which the beam deflection is greater than the deflection threshold.

FIG. 16 is a perspective view of a medical device according to an embodiment.

FIG. 17 is a side view of a portion of the medical device of FIG. 16 according to an embodiment.

FIG. 18 is a cross-sectional view of the medical device of FIG. 16 taken at N-N.

FIG. 19 is a perspective view of a medical device according to an embodiment.

FIG. 20 is a cross-sectional view of the medical device of FIG. 16 taken at P-P.

FIG. 21 is a diagrammatic illustration of a portion of a medical instrument according to an embodiment.

FIG. 22A is a graph showing strain (Y-axis) as a function of the longitudinal positions (X-axis) along the beam during operation of the medical instrument of FIG. 21 on a condition in which the beam deflection is less than a deflection threshold.

FIG. 22B is a graph showing strain (Y-axis) as a function of the longitudinal positions (X-axis) along the beam during operation of the medical instrument of FIG. 21 on a condition in which the beam deflection is greater than the deflection threshold.

FIG. 23 is a schematic illustration of a controller for use with a minimally invasive teleoperated surgery system according to an embodiment.

FIG. 24 is a flow chart of a method of control for a surgical system according to an embodiment.

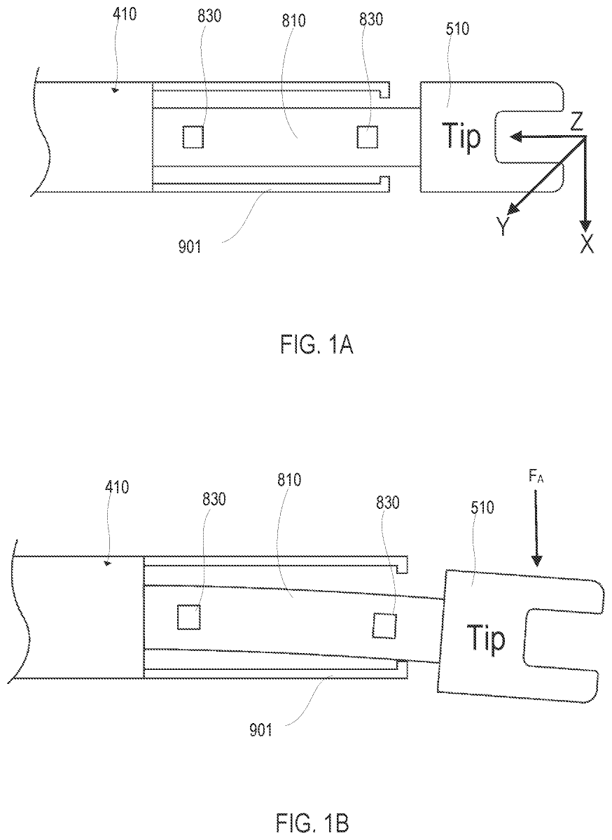

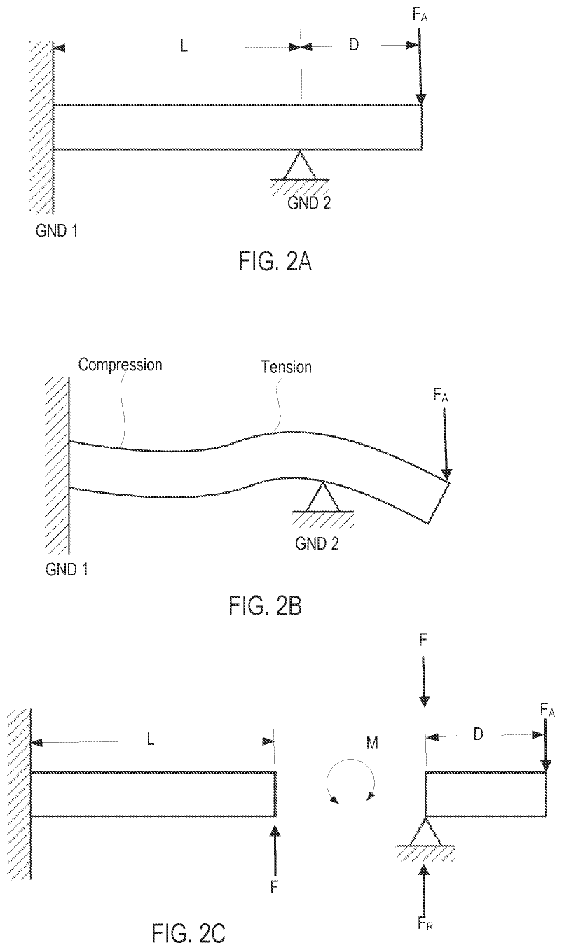

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

The embodiments described herein can advantageously be used in a wide variety of operations associated with minimally invasive surgery, including grasping, cutting, and otherwise manipulating tissue. The medical instruments or devices of the present application enable motion in three or more degrees of freedom (DOFs). For example, in some embodiments, an end effector of the medical instrument can move with reference to the main body of the instrument in three mechanical DOFs, e.g., pitch, yaw, and roll (shaft roll). There may also be one or more mechanical DOFs in the end effector itself, e.g., two jaws, each rotating with reference to a clevis (2 DOFs), and in turn the clevis may optionally rotate with reference to a more proximal clevis (one DOF) or other mechanical reference. Thus, in some embodiments, the medical instruments or devices of the present application may enable motion in six or more DOFs, including all six Cartesian DOFs. Further, the embodiments described herein are used to deliver a modified force feedback to a system operator in response to forces exerted on (or by) a distal end portion of the instrument during use under certain operating conditions.

Persons of skill in the art will understand that during surgery, various forces will be applied to a surgical instrument's distal end. In some situations the applied force may directly act on the instrument, in some situations the applied force may be a reactive force as a result of the instrument acting on another object, and in some situations, the applied force may be a combination of a direct action force and a reactive force. The weight of retracted tissue and another instrument striking the instrument are examples of forces directly acting on the instrument. Alternatively, the force exerted by resilient or hard tissue or when tightening suture are examples of reactive forces acting on the instrument as it moves against these objects. In this description, any one of the directly acting, reactive, or combined direct and reactive forces are referred to as an applied force on the instrument. When using a hand-operated instrument, the clinician experiences this applied force as a direct haptic sensation through the instrument. But when using a motor-powered instrument, this applied force is isolated from the human clinical operator, and so the applied force is detected, measured, and fed back to the clinical operator via a haptic force feedback system.

Generally, the present disclosure is directed to systems and methods for controlling a surgical system such as a minimally invasive teleoperated surgery system. In particular, the present disclosure may include a system and methods that may facilitate the delivery of haptic to the operator of the surgical system based on a determined magnitude of an applied force affecting a medical instrument. The applied force can be determined (e.g., measured) by a force sensor unit of the medical instrument. In a first condition, the magnitude of the applied force may be such that the resultant deflection of a beam of the force sensor unit is determined only by the mechanical properties of the beam under the given operating conditions. In this condition, the output of strain sensor of the force sensor unit (e.g., a determined force, also known as a “measured force”) corresponds to the applied force. However, in a second condition, the magnitude of the applied force results in a deflection of the beam that brings the beam into contact with a relatively more rigid structure that limits further deflection of the beam. The contact between the beam and the more rigid structure results in the development of a reactive force in the direction opposite the applied force. As such, the output of strain sensor (e.g., the determined force) does not accurately indicate the magnitude of the applied force in the second condition. Therefore, it is desirable to detect when the portion of the instrument is in contact with the more rigid structure due to the deflection of the portion of the instrument.

As described herein, it is desirable to determine the impact of the reactive force in order to determine the magnitude of the applied force. For example, the magnitude of the reactive force can be directly measured via a strain sensor that is coupled to the relatively more rigid structure. Alternatively, an additional strain sensor can be coupled to the beam and a known point of contact between the beam and the relatively more rigid structure can be established at a fixed longitudinal position. The known longitudinal positions of the strain sensors and the point of contact can then be utilized with the strain indications from the strain sensors to determine strain functions that are indicative of the magnitude of the applied force. As such, the systems and methods disclosed herein facilitate the determination of the magnitude of the applied force and the generation of haptic feedback delivered to the operator of the surgical system.

As used herein, the term “about” when used in connection with a referenced numeric indication means the referenced numeric indication plus or minus up to 10 percent of that referenced numeric indication. For example, the language “about 50” covers the range of 45 to 55. Similarly, the language “about 5” covers the range of 4.5 to 5.5.

The terms “flexible” or “rigid” in association with a part, such as a mechanical structure, component, or component assembly, should be broadly construed. In essence, the terms refer to the attributes of the part that determine whether the part can be repeatedly bent and restored to an original shape without harm to the part. Many “rigid” objects can have a slight inherent resilient “bendiness” due to material properties, although such objects are not considered “flexible” as the term is used herein.

As used in this specification and the appended claims, the word “distal” refers to direction towards a work site, and the word “proximal” refers to a direction away from the work site. Thus, for example, the end of a tool that is closest to the target tissue is the distal end of the tool, and the end opposite the distal end (i.e., the end manipulated by the user or coupled to the actuation shaft) is the proximal end of the tool.

Further, specific words chosen to describe one or more embodiments and optional elements or features are not intended to limit the invention. For example, spatially relative terms—such as “beneath”, “below”, “lower”, “above”, “upper”, “proximal”, “distal”, and the like—may be used to describe the relationship of one element or feature to another element or feature as illustrated in the figures. These spatially relative terms are intended to encompass different positions (i.e., translational placements) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the term “below” can encompass both positions and orientations of above and below. A device may be otherwise oriented (e.g., rotated 90 degrees or at other orientations) and the spatially relative descriptors used herein interpreted accordingly. Likewise, descriptions of movement along (translation) and around (rotation) various axes includes various spatial device positions and orientations. The combination of a body's position and orientation define the body's pose.

Similarly, geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

In addition, the singular forms “a”, “an”, and “the” are intended to include the plural forms as well, unless the context indicates otherwise. The terms “comprises”, “includes”, “has”, and the like specify the presence of stated features, steps, operations, elements, components, etc. but do not preclude the presence or addition of one or more other features, steps, operations, elements, components, or groups.

Aspects of the invention are described using a da Vinci® surgical system (commercialized by Intuitive Surgical, Sunnyvale, California) as an example surgical system form. Knowledgeable persons will understand that inventive aspects disclosed herein may be embodied and implemented in various ways, including computer-assisted, non-computer-assisted, and hybrid combinations of manual and computer-assisted embodiments and implementations. Implementations on da Vinci® Surgical Systems are not to be considered as limiting the scope of the inventive aspects disclosed herein. As applicable, inventive aspects may be embodied and implemented in both relatively smaller, hand-held, hand-operated devices and relatively larger systems that have additional mechanical support.

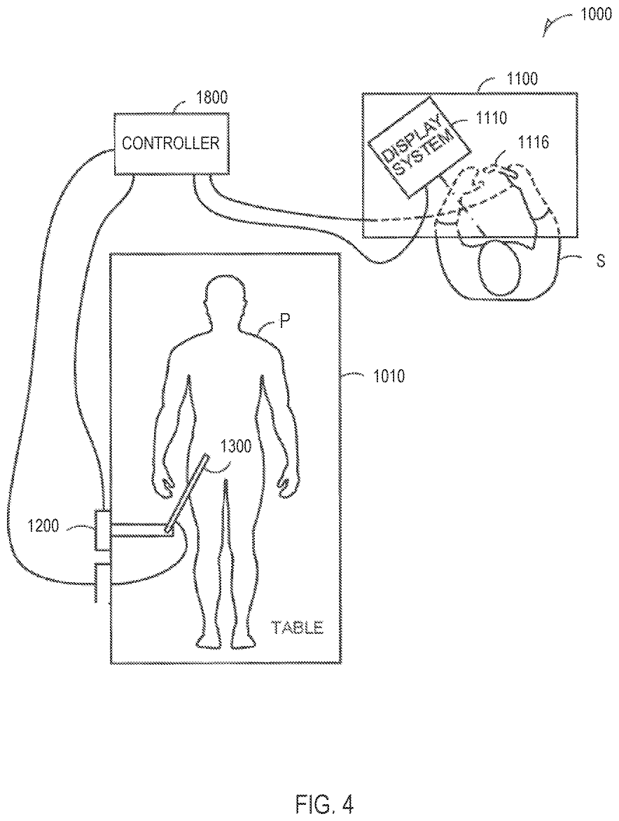

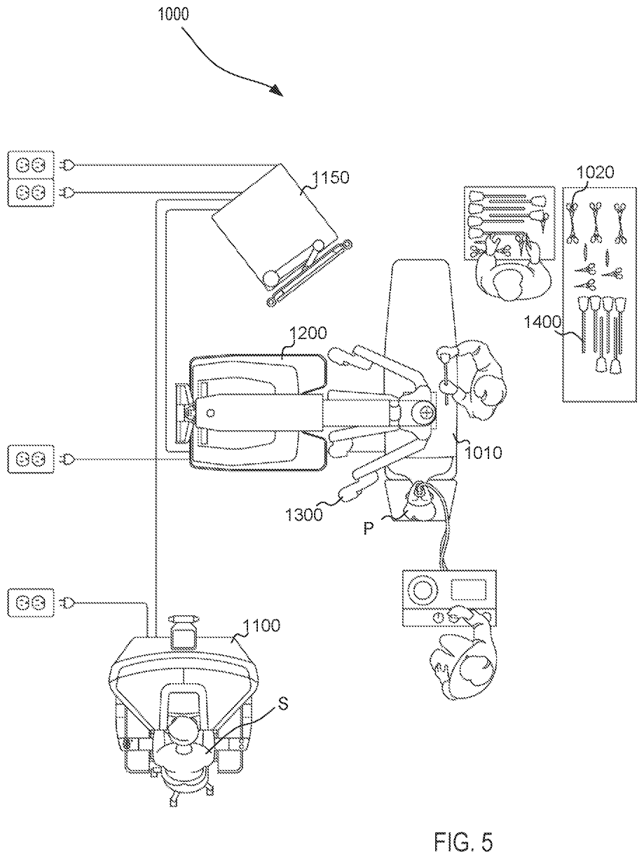

FIGS. 4 and 5 are plan view illustrations of a teleoperated surgical system 1000 that operates with at least partial computer assistance (a “telesurgical system”). Both the telesurgical system 1000 and its components are considered medical devices. The telesurgical system 1000 is a Minimally Invasive Robotic Surgical (MIRS) system used for performing a minimally invasive diagnostic or surgical procedure on a Patient (P) who is lying on an Operating table 1010. The system is made of various optional components, such as a user control unit 1100 for use by a surgeon or other skilled clinician S (e.g., operator of the surgical system) during the procedure. The telesurgical system 1000 further includes a manipulator unit 1200 (popularly referred to as a surgical robot) and an optional auxiliary equipment unit 1150. The manipulator unit 1200 includes an arm assembly 1300 and an instrument (e.g., a surgical instrument tool assembly) optionally removably coupled to the arm assembly. The manipulator unit 1200 can manipulate at least one removably coupled instrument 1400 through a minimally invasive incision in the body or natural orifice of the patient (P) while the surgeon S views the surgical site and controls movement of the instrument 1400 through control unit 1100 with the assistance of a controller 1800. Further details of the controller 1800 are described below with reference to FIG. 27. An image of the surgical site is obtained by an endoscope, such as a stereoscopic endoscope, which can be manipulated by the manipulator unit 1200 to orient the endoscope. The auxiliary equipment unit 1150 can be used to process the images of the surgical site for subsequent display to the surgeon S via a display system 1110 of the user control unit 1100. The number of instruments 1400 used at one time will generally depend on the diagnostic or surgical procedure and the space constraints within the operating room, among other factors. If it is necessary to change one or more of the instruments 1400 being used during a procedure, an assistant removes the instrument 1400 from the manipulator unit 1200 and replaces it with another instrument 1400 from a tray 1020 in the operating room. Although shown as being used with the instruments 1400, any of the instruments described herein can be used with the telesurgical system 1000. It should be appreciated that the surgical site is either at the skin surface or within at least a portion of the body of the patient (P).

The user control unit 1100 is shown in FIGS. 4 and 5 as being in the same room as the patient (P) so that the surgeon S can directly monitor the procedure, be physically present if necessary, and speak to an assistant directly rather than over the telephone or other communication medium. In other embodiments, however, the user control unit 1100 and the surgeon S can be in a different room, a completely different building, or other location remote from the patient (P), allowing for remote surgical procedures.

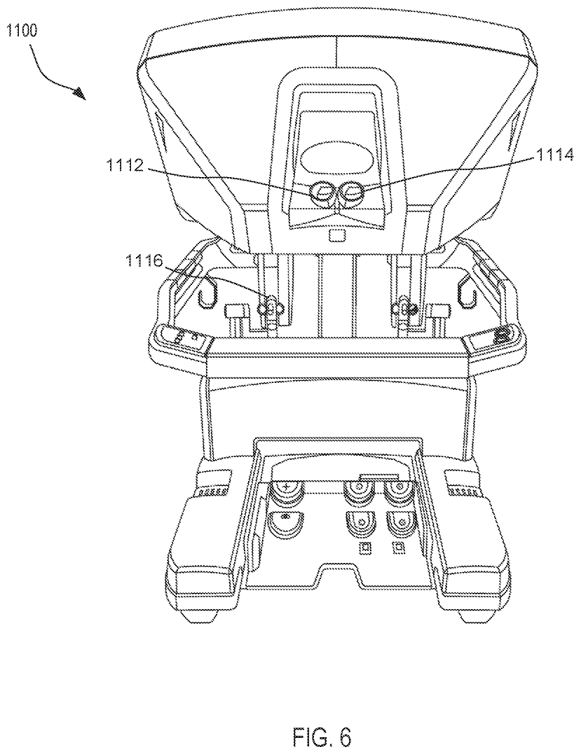

FIG. 6 is a perspective view of the control unit 1100. The user control unit 1100 includes one or more input control devices 1116 configured to be held by the surgeon S, which in turn cause the manipulator unit 1200 to manipulate one or more instrument (e.g., tools, medical devices, and/or surgical instruments). The input control devices 1116 provide at least the same degrees of freedom as instruments 1400 with which they are associated to provide the surgeon S with telepresence, or the perception that the input control devices 1116 are integral with the instruments 1400. In this manner, the user control unit 1100 provides the surgeon S with a strong sense of directly controlling the instruments 1400. To this end, impressions (e.g., haptic feedback) of position, force, strain, or tactile feedback sensors (not shown) or any combination of such sensations, are delivered from the instruments 1400 back to the surgeon S through the one or more input control devices 1116.

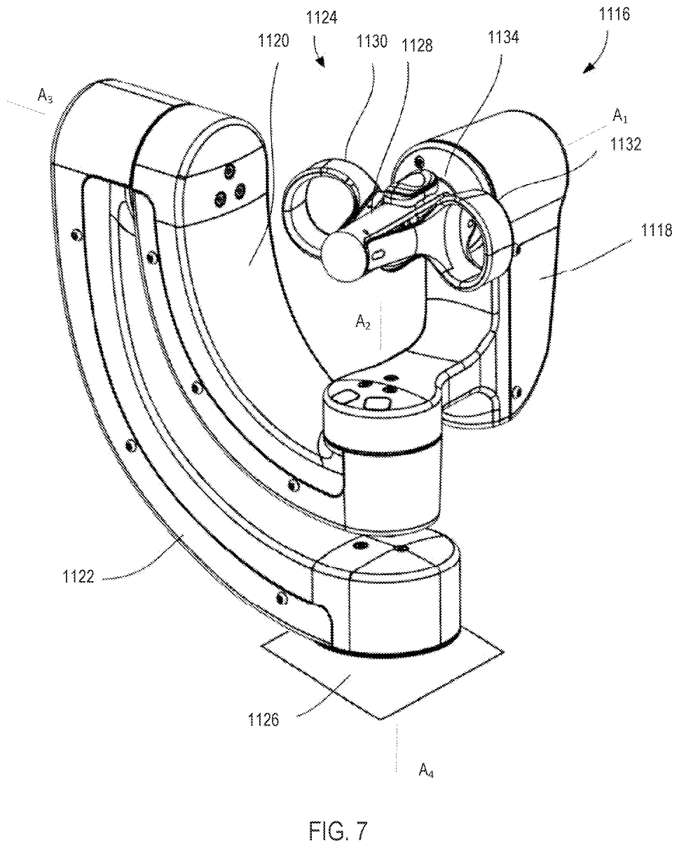

FIG. 7 is a perspective view of an input control device 1116 configured to be held with at least a portion of surgeon's S hand, according to an embodiment. In such a configuration, links are interconnected in a gimbal arrangement so that the input control device 1116 includes a first link 1118 (which functions as a first gimbal link), a second link 1120 (which functions as a second gimbal link), a third link 1122 (which functions as a third gimbal link), and an input handle 1124. The input control device 1116 is mounted to a base portion 1126, which is a distal portion of an kinematic arm that itself is a part of a user control unit, such as the user control unit 1100 described herein. Although an individual mechanically grounded hand input control device is shown for illustrative purposes, other optional gimbal and non-gimbal configurations, and mechanically grounded and ungrounded configurations, are known and may be used in accordance with the inventive aspects described herein.

As shown, the input handle 1124 includes a handle portion 1128, an optional first grip lever input 1130, an optional second grip lever input 1132, and a handle input shaft 1134. In an embodiment, the handle input shaft 1134's long axis defines a first rotational axis A₁ (which in this description functions as a roll axis; the term roll is arbitrary) and is rotatably coupled to the first link 1118. The handle portion 1128 is supported on the handle input shaft 1134 and is configured to be rotated relative to the first link 1118 about the first rotational axis A₁. The input shaft 1134 extends at least partially within the first link 1118. The first handle input 1130 and the second handle input 1132 can be manipulated to produce a desired action at the instrument end effector (not shown) operatively coupled to the input device 1116 and its handle 1124. For example, in some embodiments, the first grip lever input 1130 and the second grip lever input 1132 can be squeezed together to produce a gripping movement at the end effector. The first and second grip lever inputs 1130, 1132 are similar to the grip members shown and described in U.S. Patent Application Pub. No. US 2020/0015917 A1 (filed Jun. 14, 2019), entitled “Actuated Grips for Controller,” which is incorporated herein by reference in its entirety for all purposes. In other embodiments, however, the input handle 1124 need not include the grip lever inputs, or the grip lever inputs are illustrative of other optional hand-operated control inputs (e.g., buttons, levers, switches, wheels) that may be used in other configurations.

As depicted in FIG. 6, in an embodiment, at least one of the user control unit 1100 may be configured to be engaged via a portion of at least one foot of the surgeon S. In such a configuration, the user control unit 1100 can include at least one pedal assembly 1136 and/or at least one foot-activated switch assembly 1138. Each pedal assembly 1136 and/or foot-activated switch assembly 1138 may include at least one switch (not shown) activated by the respective assembly. The surgical system 1000 may detect that one or more electrosurgical tools are mounted to the manipulator unit 1200 and may assign the appropriate control functions to the pedal assembly 1136 and/or foot-activated switch assembly 1138.

In some embodiments, the user control unit 1100 includes one or more optional touchpads 1140 configured to receive an input from the surgeon S. The touchpad(s) 1140 may, for example, be a liquid crystal display (LCD) screen. The touchpad(s) 1140 may, as depicted in FIG. 6, be mounted in an arm rest or at another suitable location of the user control unit 1100. The surgeon S may utilize the touchpad(s) 1140 to access various operations, protocols, and/or settings of the surgical system 1000, such as user accounts, ergonomic settings, preferences, equipment configurations, operational status commands, and/or other similar processes as described herein. Additionally, the human clinical operator may use the touchpad(s) 1140 to acknowledge various system messages, alerts, and/or warnings as described herein.

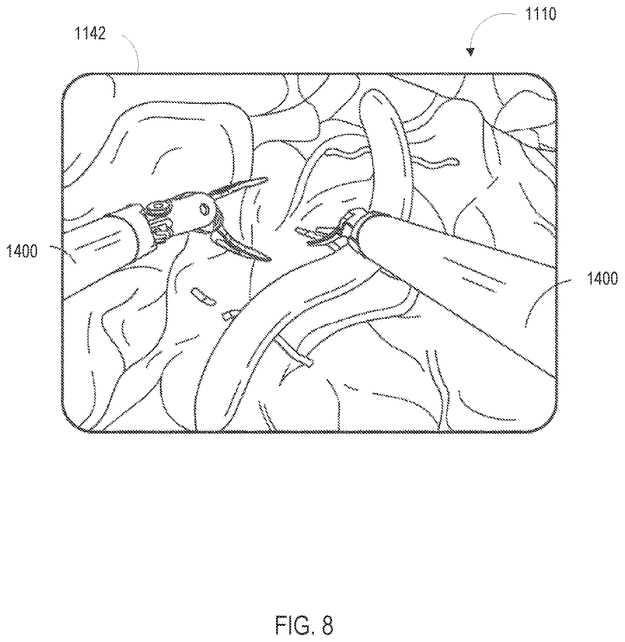

As further depicted in FIG. 6, the user control unit 1100 includes a display system 1110. In other user control unit examples, the display is separate from the console structure and may be, for example, mounted on a wall or other support structure. As depicted in FIG. 8, the display system 1110 defines a field-of-view 1142 of the operator S. In some embodiments, the display system 1110 is stereoscopic and includes a left eye display 1112 and a right eye display 1114 for presenting the surgeon S with a coordinated stereoscopic view of the surgical site that enables depth perception. In other embodiments a monoscopic display may be used. Various other stereoscopic and monoscopic display systems are known and are contemplated to be within the scope of various inventive aspects described herein. True three-dimensional displays are contemplated. Such stereoscopic and monoscopic display systems may be mechanically grounded as shown, or they may be mechanically ungrounded and embodied in devices such as head mounted displays. Although not shown in FIG. 8, it is well understood that display system 1110 may optionally display various messages to the operator that include information aspects as described herein (e.g., information about the status of a haptic feedback system).

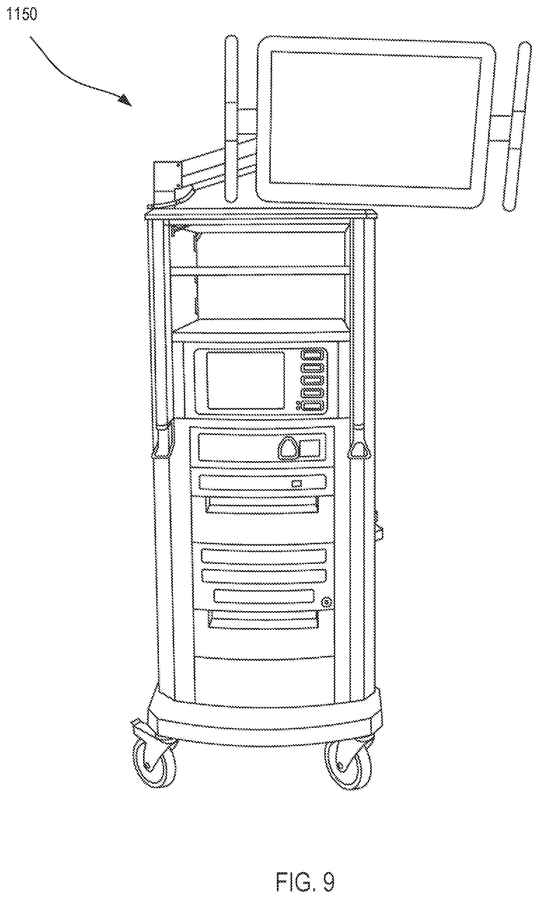

FIG. 9 is a perspective view of the auxiliary equipment unit 1150. In some embodiments the auxiliary equipment unit 1150 is coupled with the endoscope and includes one or more processors to process captured images for subsequent display, such as via the display system 1110 of the user control unit 1100, or on another suitable display located locally (e.g., on the unit 1150 itself as shown, on a wall-mounted display) and/or remotely. For example, if a stereoscopic endoscope is used, the auxiliary equipment unit 1150 processes the captured images to present the surgeon S with coordinated stereo images of the surgical site via the left eye display 1112 and the right eye display 1114. Such coordination optionally includes alignment between the opposing images and optionally includes adjusting the stereo working distance of the stereoscopic endoscope. As another example, image processing can include the use of previously determined camera calibration parameters to compensate for imaging errors of the image capture device, such as optical aberrations.

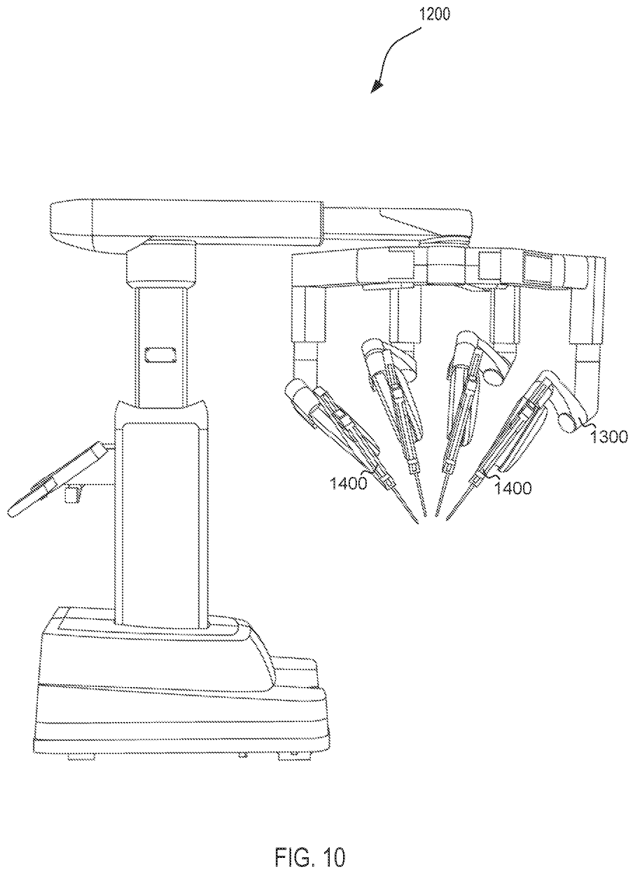

FIG. 10 is a side view of the manipulator unit 1200. The manipulator unit 1200 includes the components (e.g., arms, linkages, motors, sensors, and the like) to provide for the manipulation of the instruments 1400 and an imaging device (e.g., the endoscope) used for the capture of images of the site of the procedure. Specifically, the instruments 1400 and the imaging device can be manipulated by teleoperated mechanisms having one or more mechanical joints. Moreover, the instruments 1400 and the imaging device are positioned and manipulated through incisions or natural orifices in the patient (P) in a manner such that a center of motion remote from the manipulator and typically located at a position along the instrument shaft is maintained at the incision or orifice by either kinematic mechanical or software constraints. In this manner, the incision size can be minimized and tissue damage minimized at the incision.

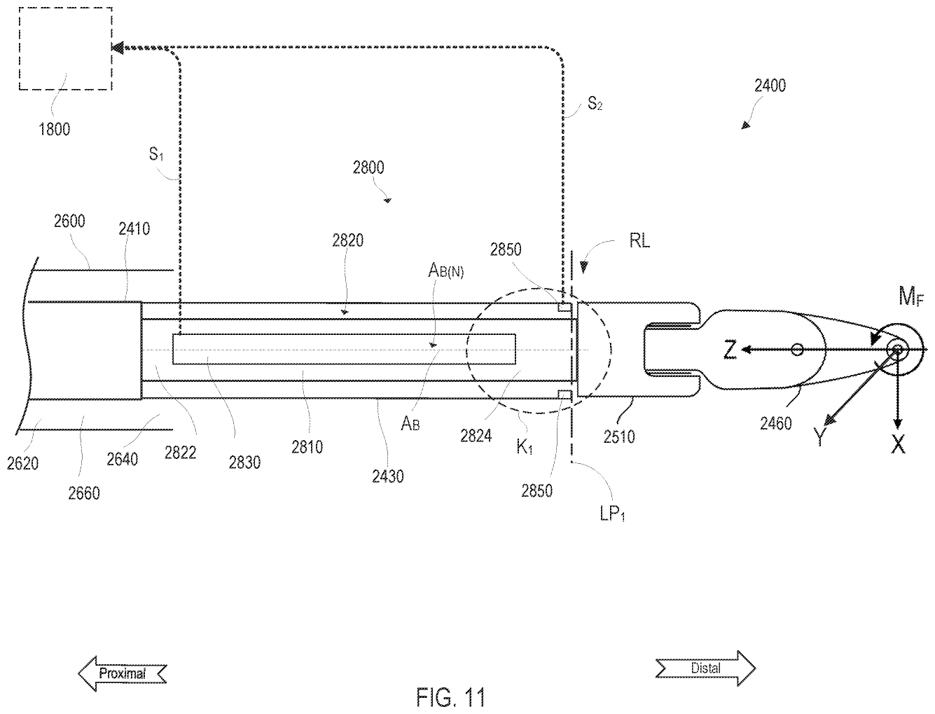

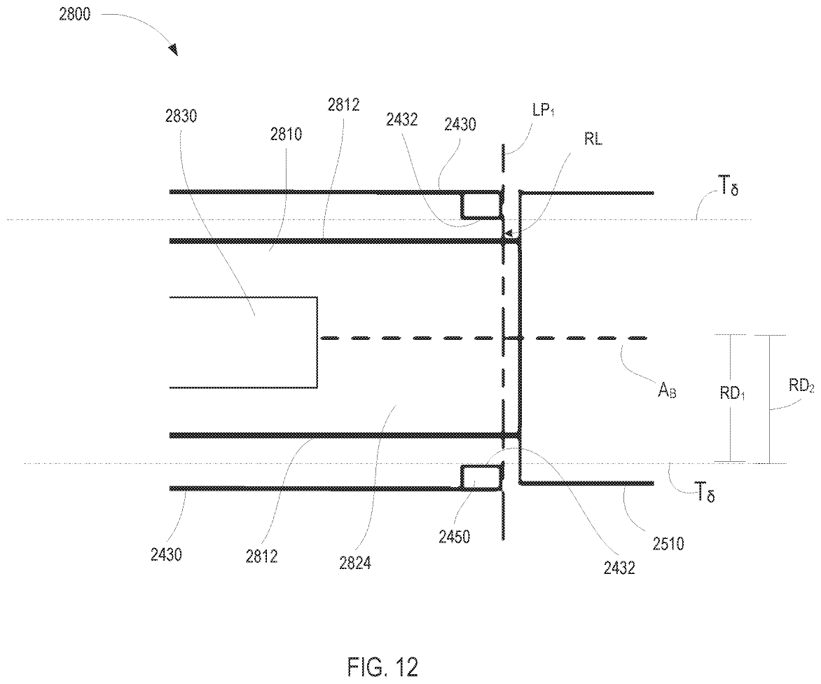

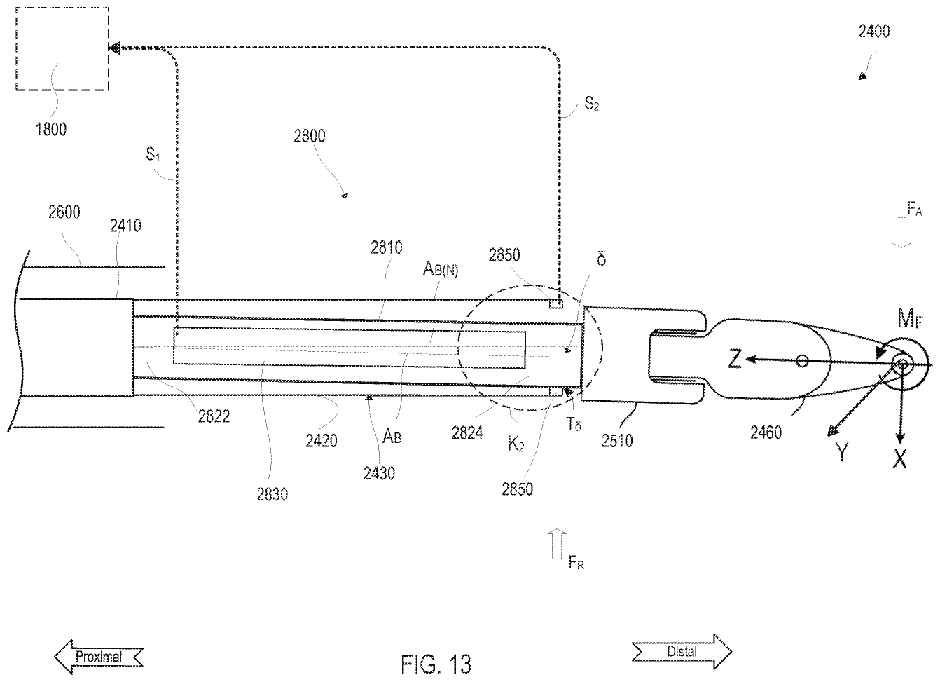

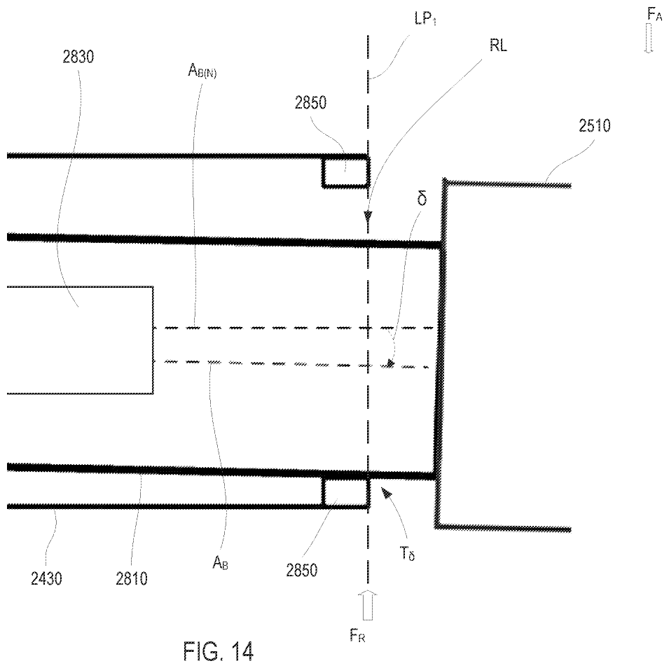

FIG. 11 is a schematic illustration of a distal end portion of an instrument 2400, such as instrument 1400 employed with the surgical system 1000, according to an embodiment. FIG. 12 is an enlarged view of a portion of the instrument 2400 of FIG. 11 indicated by the region K₁. FIG. 13 is a schematic illustration of the distal end portion of the instrument 2400 in a deflected state resulting from an applied force. FIG. 14 is an enlarged view of a portion of the instrument 2400 of FIG. 12 indicated by the region K₂. As depicted, the instrument 2400 extends through a cannula structure 2600, and a portion of the instrument 2400 is surrounded by cannula structure 2600. The cannula structure 2600 has a proximal end 2620 and a distal end 2640. The cannula structure 2600 has a central channel 2660 that extends between the proximal end 2620 and the distal end 2640, through which the instrument 2400 is inserted, such as during a medical procedure. The cannula structure 2600 can be a straight cannula as shown. In additional embodiments, the cannula structure 2600 can optionally be a curved cannula having a combination of linear and nonlinear sections, a cannula with multiple non-parallel linear sections, a cannula with multiple curve sections having different characteristics, and/or a cannula with other combinations of linear and nonlinear sections. The cannula structure 2600 has a mechanical ground connection (not shown) with an arm assembly (e.g., arm assembly 1300) of the surgical system.

The instrument 2400 includes a shaft 2410 and a force sensor unit 2800 (e.g., a force sensor assembly). The force sensor unit 2800 includes a resiliently deflectable (e.g., flexible) beam 2810, a first strain sensor 2830 mounted on a surface along the beam 2810, and a second strain sensor 2850. As is more fully described below, the first strain sensor 2830 can include one or more strain gauges arranged in one or more strain sensing circuits to sense strain that results from beam 2810 deflecting. The second strain sensor 2850 includes one or more strain gauges and/or for sensors positioned to measure reactive forces associated with contact between the beam 2810 and a hard stop structure 2430 that at least partially surrounds the beam 2810.

The shaft 2410 includes a distal end portion that is coupled to a proximal end portion 2822 of the beam 2810. In some embodiments, proximal end portion 2822 of the beam 2810 is directly coupled to the distal end portion of the shaft 2410, and in other embodiments the proximal end portion 2822 of the beam 2810 is coupled to the proximal end portion of the shaft 2410 via another coupling component (such as a mechanical anchor or coupler, not shown). In some embodiments a proximal end portion of the shaft 2410 is coupled to a mechanical structure (not shown) configured to move one or more components of the surgical instrument, such as, for example, the end effector 2460. In other words, in some embodiments, the shaft 2410 has a mechanical ground connection with the arm assembly (e.g., via an instrument carriage) of the surgical system. Thus, the beam 2810 couples the connecting link 2510 (and the end effector 2460) to the shaft 2410 in a cantilevered configuration anchored at the proximal end portion 2822 of the beam.

One or more distal end components of the instrument 2400 (e.g., a surgical end effector, a wrist assembly, and the like) are connected to the distal end portion 2824 of the beam 2810 via the connecting link 2510. As shown, an example end effector 2460 may by coupled at a distal end portion 2824 of the beam 2810 (i.e., at a distal end portion of the instrument 2400). The end effector 2460 can include, for example, articulatable jaws, a cautery instrument, and/or any other suitable surgical tool that is coupled to a link 2510 (e.g., a proximal clevis pin). In some embodiments, the link 2510 can be included within a wrist assembly having multiple articulating links. In some embodiments the link 2510 is included as part of the end effector 2460.

As shown, the beam 2810 of the force sensor unit 2800 includes a middle portion 2820 that is between a proximal end portion 2822 and a distal end portion 2824. The beam 2810 center axis A_{B} extends longitudinally, is defined by end portions 2822, 2824, and is centered on the beam. In some embodiments center axis A_{B} is aligned (collinear) with a similar center axis (not shown) of the instrument shaft 2410, and in other embodiments these two axes are not collinear. As described below, in the absence of deflection, the axis AB response is in a designed position A_{B(N)}. However, the deflection of the beam from axis AB from the designed position A_{B(N)} (as measured by the first strain sensor 2830) can be correlated to applied forces applied to the end effector 2460.

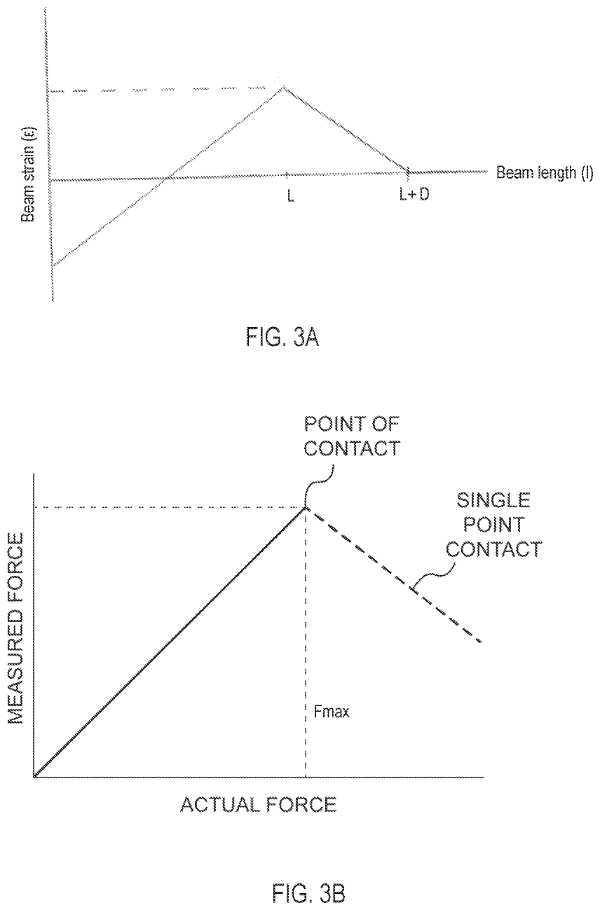

Generally, Cartesian X, Y, and Z direction forces (direct or reactive) are imparted on the end effector 2460. In practice, an applied force can be deconstructed into its Cartesian components. Resolved moments M_{F} corresponding to these X, Y, or Z forces are likewise imparted to the end effector, and the magnitude of such moments depends on a defined origin for a moment. For example, as shown in FIG. 3 an applied force F_{A} in the X direction results in a resolved moment M_{F} about the Y-direction axis. More specifically, as described herein applied forces acting generally normal to the beam center axis A_{B} (i.e., the X- and Y-direction forces as shown) will result in a corresponding moment on beam 2810. Forces acting in the Z-direction along the center axis A_{B} are a special case because they act through center axis A_{B}, and so through the defined origin with a resulting moment of zero magnitude.

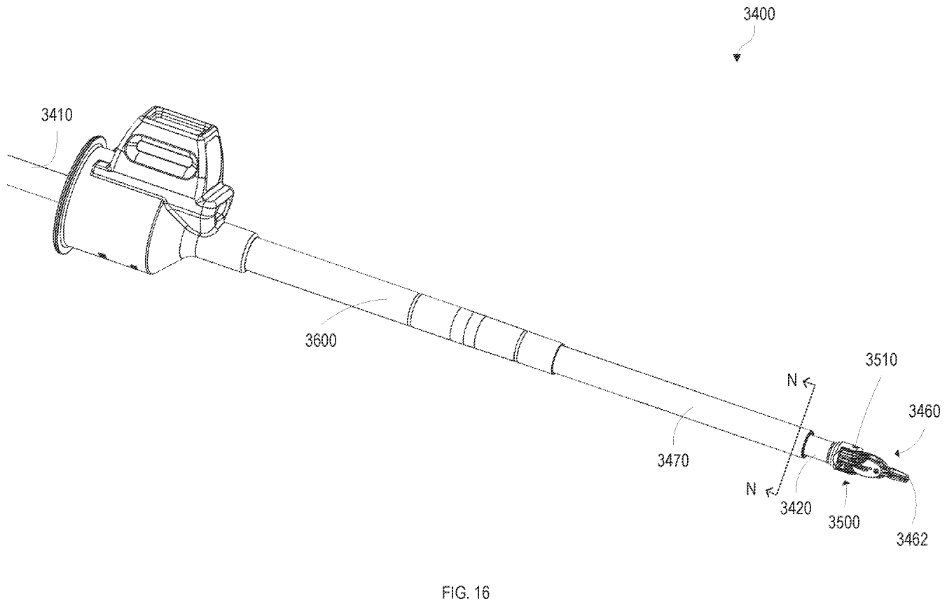

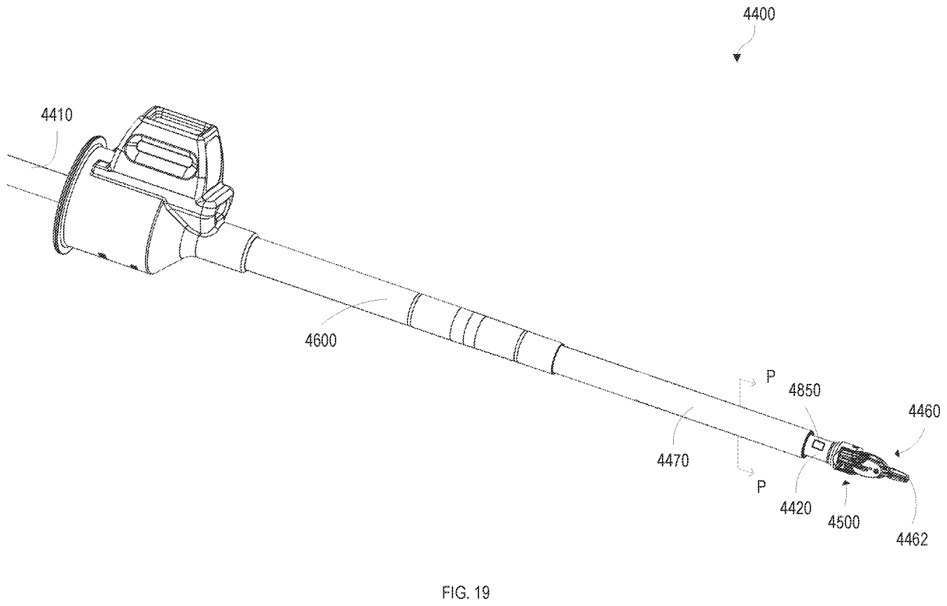

In some embodiments, a hard stop structure 2430 at least partially surrounds the beam 2810. The hard stop structure 2430 can have a rigidity (e.g., stiffness) that is greater than the rigidity of the beam 2810. Accordingly, the hard stop structure 2430 can limit or restrict the deflection of the beam 2010 in response to an applied force when the deflection of the beam 2810 brings the beam 2810 into contact with the hard stop structure 2430. In some embodiments, the hard stop structure 2430 can be at least one of a shroud, an outer shaft, and/or the cannula structure 2600 (e.g., shrouds 3420, 4420, outer shafts 3470, 4470, and/or cannulas 3600, 5600 in FIGS. 16 and 19). The hard stop structure 2430 includes a reference location RL at which the distal end portion 2824 of the beam 2810 contacts the hard stop structure 2430 when the deflection δ of the beam 2810 is larger than the deflection threshold T_{δ}. Said another way, in some embodiments, beam deflection is limited by a hard stop location. For example, the shroud, the outer shaft, and/or the cannula structure can limit the displacement of the beam 2810. Such deflection limiting produces a reactive force F_{R} on the beam 2810. In some embodiments, the instrument can include one or more hard stop structures that function as a deflection limiting hard stop structure 2430. It should be appreciated that the hard stop structure 2430 can include known point of contact (e.g., the reference location RL) between structure of the instrument and/or a structure coupled to the instrument 2400 at a specified longitudinal location to limit deflection of the beam 2810.

As contact between the beam 2810 and the hard stop structure 2430 can affect force indications from the first strain sensor 2830, the deflection threshold T_{δ} is established in some embodiments. As depicted in FIG. 12, the deflection threshold T_{δ} can be established at a magnitude of deflection δ that is greater than zero but that precludes contact between an outer surface 2812 of the beam 2810 and a face 2432 (e.g., a radially inner face of the hard stop structure 2430 or the second strain sensor 2850). For example, the deflection threshold T_{δ} can correspond to a first radial distance (RD₁) from the designed position A_{B(N)} of the center axis A_{B} that is less than a second radial distance (RD₂) (e.g., minimal radial distance) of the face 2432 from the designed position A_{B(N)} of the center axis A_{B}. However, in some embodiments, the first radial distance (RD₁) can equal the second radial distance (RD₂) so that the deflection threshold T_{δ} corresponds to a deflection δ that places the outer surface 2812 in contact with the face 2432 but precludes the hard stop structure 2430 from exerting a reactive force on the beam 2810. In some embodiments, the deflection threshold T_{δ} corresponds to a deflection δ that places the outer surface 2812 in substantial contact with the face 2432 such that the hard stop structure 2430 limits the deflection of the beam 2810.

To sense strain that results from beam 2810 deflecting, the first strain sensor 2830 optionally includes one or more electrical strain sensing circuits (e.g., bridge circuits), and other strain sensor configurations are contemplated (e.g., optical fiber Bragg grating sensors, piezoelectric sensors, and the like). As described herein, each bridge circuit (and also each strain sensor) includes one or more strain gauges (e.g., tension strain gauge resistor(s) or compression strain gauge resistor(s)). It should be appreciated that the beam 2810 can include any number of first strain sensor 2830 in various arrangements.

A first strain sensor 2830 can, for example, include at least four strain gauge resistors arranged into one or more bridge circuits (e.g., a Wheatstone bridge). The strain gauge resistors can measure strain in the beam 2810 that can be used to determine the forces imparted on the end effector 2460 in the X and Y axis directions normal to the beam. These X and Y axes forces are transverse (e.g., perpendicular) to the Z axis (which is parallel or collinear with the longitudinal center axis A B of the beam 2810). As depicted in FIG. 13-14, such transverse forces acting upon the end effector 2460 cause a deflection (e.g., bending) of the beam 2810 (about either or both of the X axis or the Y axis), which because of its cantilever configuration results in a tensile strain imparted to one side of the beam 2810 and a compression strain imparted to the opposite side of the beam 2810. The strain gauge resistors of the first strain sensor 2830 can be used to determine such tensile and compression strains.

It will be understood that the exact location of an applied force (or force component) on the instrument is not determined, because the location along the length of the instrument at which the force (or force component) is applied is unknown. For example, a force applied directly to the distal tip of the end effector will cause the same sensed strain as a slightly larger force applied a short distance proximal of the end effector's distal tip due to an infinite number of force and moment pairs that would result in the same strain. This situation is also true for hand-operated instruments. In practice, this determined force effectively mimics a similar condition in a hand-held instrument and can be effectively used in a haptic force feedback system for a human clinical operator because of the relatively small differences in applied force locations at the instrument's distal end portion.

A first signal S₁ generated by the first strain sensor 2830 is associated with the deflection δ of the beam 2810. In other words, the outputs of the strain gauge resistors of the first strain sensor 2830 on the beam 2810 (e.g., the first signal S₁) is correlated to a determined force F. Moreover, as described herein, depending on the arrangement of the strain gauge resistors (i.e., in two half-bridge circuits), the strain gauge resistors can be used to determine the moment that results from an applied force on the instrument. It should be appreciated that the output of the force sensor unit 2800 may be used by a controller, such as the controller 1800 of system 1000 described above, to determine the haptic feedback to deliver to the surgeon S via the control unit 1100.

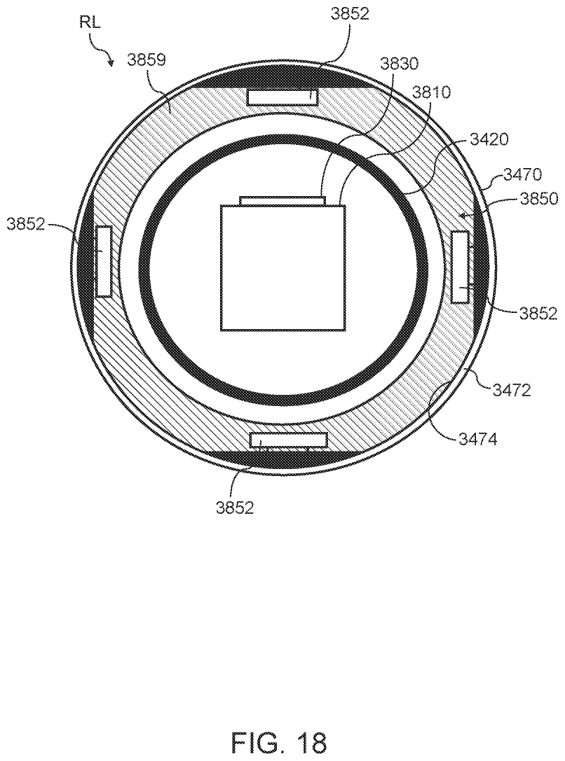

As depicted, the force sensor unit 2800 includes the second strain sensor 2850. The second strain sensor is positioned to measure reactive forces associated with contact between the beam 2810 and the hard stop structure 2430. As depicted in FIGS. 11-14, in some embodiments, the second strain sensor 2850 is positioned on the hard stop structure 2430. However, in some embodiments as described below, the second strain sensor 2850 can be coupled to the beam 2810. The second strain sensor 2850 can include at least one strain gauge resistor, at least one bridge circuit, and/or a force sensor (e.g., force sensor 3852 depicted in FIG. 18). The force sensor can, for example, be a load cell, a force transducer, a force sensing resistor, an optical force sensor, an ultrasonic force sensor, or a combination thereof. In some embodiments, such as depicted in FIGS. 11-14, the second strain sensor 2850 can be positioned at a longitudinal position that corresponds to the reference location RL.

A second signal S₂ of the second strain sensor 2850 is indicative of whether the distal end portion 2824 of the beam 2810 is in contact with the hard stop structure 2430 at the reference location RL (e.g., at a hard stop location). For example, when the deflection δ is less than the deflection threshold T_{δ}, as depicted in FIGS. 11-12, the second signal S₂ has a magnitude of zero, which indicates an absence of contact between the beam 2810 and the hard stop structure 2430. However, when the deflection δ is less than the deflection threshold T_{δ}, as depicted in FIGS. 13-14, the magnitude of the second signal S₂ corresponds to the magnitude of the reactive force F_{R}.

Although shown as including only the force sensor unit 2800, in some embodiments, the instrument 2400 (or any of the instruments described herein) optionally include additional force sensor units to measure the axial force(s) (i.e., in the direction of the Z-axis parallel to the beam center axis A_{B} imparted on the end effector 2460. An axial force sensor unit in an example surgical instrument can comprise a deflectable planar diaphragm sensor that deflects in response to a force. Alternatively, a deflectable ferrite core can be used within an inductive coil may be used or a or a fiber Bragg grating formed within an optical fiber can be used, for example. Other axial force sensor units may be used to sense a resilient axial displacement of the shaft 2410 (e.g., relative to the proximally mounted mechanical structure, not shown). An axial force imparted to the end effector 2460 can cause axial displacement of the shaft 2410 in a direction along a center axis of the shaft (substantially parallel to the beam center axis A_{B}). The axial force may be in the proximal direction (e.g., a reactive force resulting from pushing against tissue with the end effector) or it may be in the distal direction (e.g., a reactive force resulting from pulling tissue grasped with the end effector).

In some conditions, X and/or Y forces imparted on the end effector 2460, such as depicted in FIG. 13, may result in strain in the beam 2810 when the beam 2810 is deflected δ (e.g., resiliently displaced). In other words, the X and/or Y forces deflect the center axis A_{B} of the beam 2810 away from a zero-force designed position A_{B(N)} of the center axis A_{B}, and thus deflect center axis A_{B} relative to the longitudinal center axis of the shaft 2410. Said another way, the distal end portion 2824 of the beam 2810 can bend relative to a proximal end portion 2822 of the beam 2810 such that the distal end portion 2824 of the beam 2810 is displaced a deflection distance relative to the designed position A_{B(N)} of the center axis A_{B}.

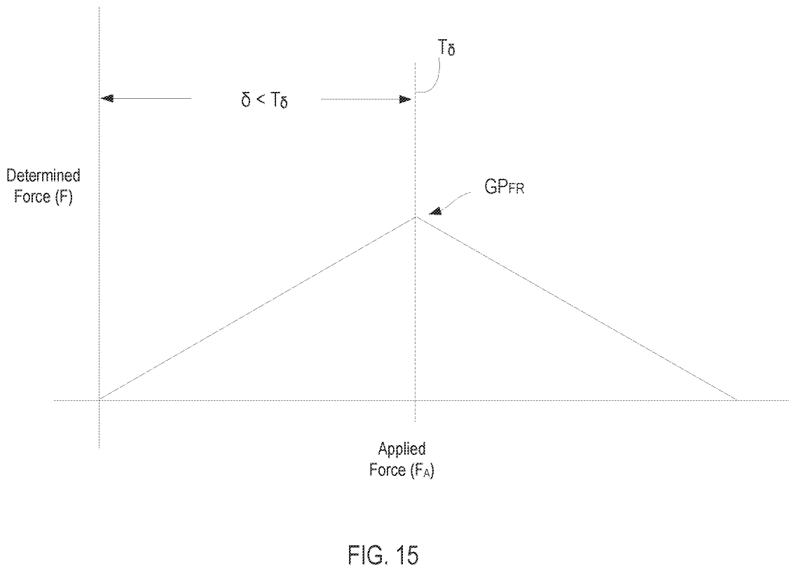

As described above, in embodiments in which the displacement of beam 2810 is limited by the hard stop structure 2430, the strain distribution over the length of the beam 2810 may deviate relative to displacements of the beam 2810 that are not limited (e.g., a deflection δ that is less than the deflection threshold T_{δ}). Similarly stated, when the beam 2810 contacts the hard stop structure 2430, the beam 2810 no longer behaves as a cantilevered beam. In this condition, the deflection δ of the beam's distal end 2824 is larger than the maximum deflection (e.g., the deflection threshold T_{δ}) permitted at the reference location RL of the hard stop structure 2430. As a result, the first strain sensor 2830 produce signals that do not accurately represent the applied force F_{A} affecting the end effector 2460, and so the force determined from the incorrect strain sensor signals (e.g., the first signal S₁) may be significantly in error. For example, as shown in FIG. 15, when the applied force F_{A} increases to a magnitude that causes a deflection δ that results in the generation of the reactive force F_{R} (the initiation of which is depicted at point GP_{FR}), the signals from the first strain sensor 2830 may result in a determined force F that is decreasing while the applied force F_{A} acting on the end effector 2460 is actually increasing (e.g., a force inversion condition may exist). Insofar as the controller (e.g., controller 1800) may use the signals from the first strain sensor 2830 to generate the haptic feedback delivered to the surgeon S, the inaccurate determination of the applied force F_{A} (i.e., an inaccurate determined force F) resulting in inaccurate haptic feedback is undesirable. It should therefore be appreciated that detecting such conditions of inaccurate determind force and mitigating the impact of inaccurate haptic feedback is beneficial to the operation of the surgical system 1000.

As depicted in FIGS. 11-14, in some embodiments the reference location RL is, in some embodiments, located at a longitudinal position LP₁ along the instrument 2400. The longitudinal position LP₁ is coplanar with the hard stop location. The reference location RL can be a portion of the hard stop structure 2430 that is opposite and facing the beam 2810. The reference location RL may, for example, be radially outward of the beam 2810 relative to the center axis A_{B}.

The positioning of the hard stop location limits deflection δ of the beam 2810. In other words, the distal end portion 2824 of the beam 2810 can bend relative to a proximal end portion 2822 of the beam 2810 such that the distal end portion 2824 of the beam 2810 is displaced a deflection distance relative to the designed position A_{B(N)} of the center axis A_{B} in response to an applied force F_{A}. However, as depicted in FIGS. 13 and 14, this bending is limited when the beam 2810 encounters the hard stop structure 2430 and/or the supporting structure being more rigid than the beam 2810.

As described herein, the first signal S₁ correlates, at least in part, to the determined force F. When the deflection δ of the beam 2810 in response to the applied force F_{A} does not result in the development of the reactive force F_{R} (e.g., when the beam 2810 does not substantially contact the hard stop structure 2430), the determined force F is effectively correlated to the applied force F_{A}. But when the deflection δ of the beam 2810 in response to the applied force F_{A} places the beam 2810 in substantial contact with the hard stop structure 2430 (as shown in FIGS. 13-14), the determined force F is the result of a combination of the applied force F_{A} and the reactive force F_{R}, which is provided by Eq. (3):

F=F_{A}−F_{R} Eq. (3)

In accordance with Eq. (3), the magnitude of the determined force F, as indicated by the first strain sensor 2830, is less than the magnitude of the actual applied force F_{A}, which is unknown, due to the reactive force F_{R}, acting on the beam 2810 in the opposite direction when the beam 2810 is in substantial contact with the hard stop structure 2430. As the second signal S₂ correlates to the reactive force F_{R}, the magnitude of the applied force F_{A} can be determined using Eq. (3). In some embodiments, the determined magnitudes of the applied force FA can be used to provide haptic feedback to a human operator of an input device (e.g., control unit 1100).

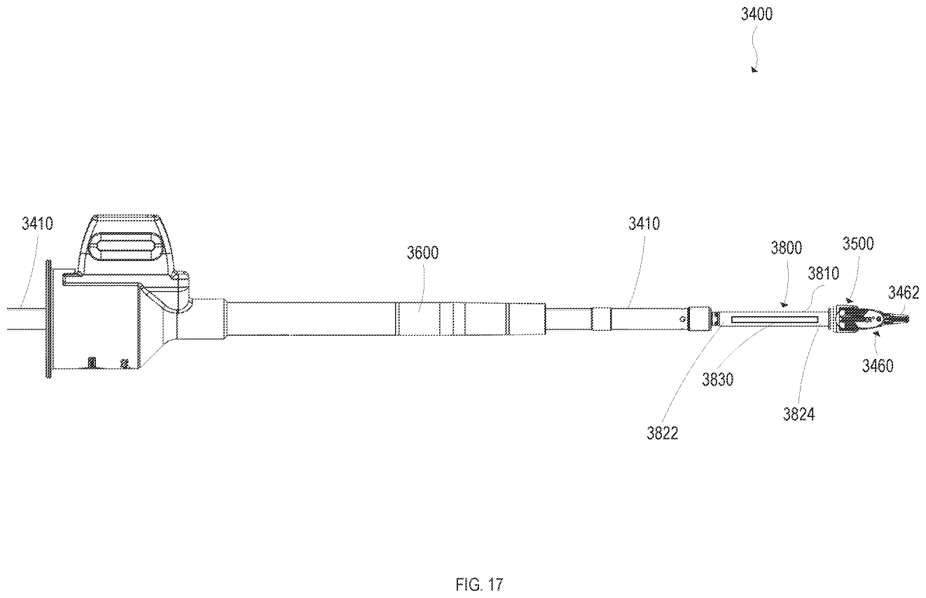

FIGS. 16 and 17 depict a perspective view and a side view (with the outer shaft and shroud removed for clarity) of a medical instrument 3400 and a cannula 3600, while FIG. 18 depicts a cross-sectional view of the of the instrument 3400 taken at N-N. In some embodiments, the instrument 3400 or any of the components thereof are optionally parts of a surgical system that performs surgical procedures. The surgical system may include a manipulator unit, a series of kinematic linkages, a series of cannulas, or the like. The instrument 3400 (and any of the instruments described herein) can be used in any suitable surgical system, such as the MIRS system 1000 shown and described above.

The instrument 3400 includes a proximal mechanical structure (not shown), an outer shaft 3470, a shaft 3410, a force sensor unit 3800, a wrist assembly 3500, and an end effector 3460. The force sensor unit 3800 includes a beam 3810, a first strain sensor 3830 coupled to the beam 3810, and a second strain sensor 3850 coupled to the outer shaft 3470. As depicted, the outer shaft 3470 can be a hard stop structure as described herein. As depicted, in an embodiment, a shroud 3420 may circumscribe at least a portion of the beam 3810. Although not shown, the instrument 3400 can also include a number of cables that couple the mechanical structure to the wrist assembly 3500 and end effector 3460. The instrument 3400 is configured such that select movements of the cables produces rotation of the wrist assembly 3500 (i.e., pitch rotation) about an axis of rotation (which functions as a pitch axis, the term pitch is arbitrary), yaw rotation of the end effector 3460 about an additional axis of rotation (which functions as the yaw axis, the term yaw is arbitrary), a cutting rotation of the tool members of the end effector 3460, or any combination of these movements. Changing the pitch or yaw of the instrument 3400 can be performed by manipulating the cables in a similar manner as described, for example, in U.S. Pat. No. U.S. 8,821,480B2 (filed Jul. 16, 2008), entitled “Four-Cable Wrist with Solid Surface Cable Channels,” which is incorporated herein by reference in its entirety.

In some embodiments, the end effector 3460 can include at least one tool member 3462 having a contact portion configured to engage or manipulate a target tissue during a surgical procedure. For example, in some embodiments, the contact portion can include an engagement surface that functions as a gripper, cutter, tissue manipulator, or the like. In other embodiments, the contact portion can be an energized tool member that is used for cauterization or electrosurgical procedures. The end effector 3460 may be operatively coupled to the proximal mechanical structure such that the tool member 3462 rotates relative to shaft 3410. In this manner, the contact portion of the tool member 3462 can be actuated to engage or manipulate a target tissue during a surgical procedure. The tool member 3462 (or any of the tool members described herein) can be any suitable medical tool member. Moreover, although only one tool member 3462 is identified, as shown, the instrument 3400 can include two tool members that cooperatively perform gripping or shearing functions. In other embodiments, an end effector can include more than two tool members.

As depicted, the end effector 3460 can be coupled to a link 3510 (e.g., a proximal clevis pin). In some embodiments, the link 3510 can be included within a wrist assembly having multiple articulating links. In some embodiments the link 3510 is included as part of the end effector 3460. The shaft 3410 includes a distal end portion that is coupled to a proximal end portion 3822 of the beam 3810. In some embodiments, the distal end portion of the shaft 3410 is coupled to the proximal end portion 3822 of the beam via another coupling component (such as an anchor or coupler, not shown). The shaft 3410 can also be coupled at a proximal end portion to a mechanical structure (not shown) configured to move one or more components of the surgical instrument, such as, for example, the end effector 3460.

In some embodiments, the first strain sensor 3830 can, for example, include any of the elements disclosed herein with reference to the first strain sensor 2830 and may be used to measure forces imparted on the surgical instrument during a surgical procedure as described in more detail herein. In some embodiments, the beam 3810 can define at least three side surfaces disposed acutely to each other. In additional embodiments, the beam 3810 can define at least four side surfaces disposed perpendicular to one another. The first strain sensor 3830 may be mounted to the side surfaces in appropriate locations. The beam 3810 defines a beam center axis (see e.g., beam center axis A_{B} described above with reference to FIGS. 11-14) which can be aligned within a center axis (not shown) of the instrument shaft 3410. The beam center axis is a neutral axis that is equidistant from the sides (e.g., faces) of the beam 3810.

In use, the end effector 3460 may contact anatomical tissue, which may result in X, Y, or Z direction forces (similar to the forces exerted on the end effector 2460 shown in FIG. 13) being imparted on the end effector 3460. This contact may also result in forces about the various axes. The first strain sensor 3830 may be used to measure strain in the beam 3810 as a result of such forces imparted on the end effector 3460. More specifically, the first strain sensor 3830 can measure forces imparted on the end effector 3460 that are transverse (e.g., perpendicular) to a center axis of the beam 3810 as such forces are transferred to the beam 3810 in the X and Y directions (see FIG. 13). Specifically, the transverse forces acting upon the end effector 3460 can cause a slight bending of the beam 3810, which can result in a tensile strain imparted to one side of the beam 3810 and a compression strain imparted to an opposing side of the beam 3810. The first strain sensor 3830 may be coupled to the beam 3810 to measure such tensile and compression forces, with the resultant measurements being communicated to the controller via a communication coupling therebetween.

More specifically, when a force is imparted on a distal portion of the instrument 3400 (e.g., at end effector 3460) in the X or Y directions (see FIG. 11B for reference to X, Y and Z directions), such transverse force can cause the beam 3810 to bend (about either or some combination of the X axis or the Y axis), which can result in a tensile strain imparted to one side of the beam 3810 and a compression strain imparted to the opposite side of the beam 3810. The first strain sensor 3830 on the beam 3810 can measure such tensile and compression strains.

Although shown as including only the force sensor unit 3800, in some embodiments, the instrument 3400 (or any of the instruments described herein) can include additional force sensor units to measure the axial force(s) (i.e., in the direction of the Z-axis parallel to the beam center axis A_{B}) imparted on the end effector 3460. An axial force sensor unit in an example surgical instrument can comprise a deflectable planar diaphragm sensor that deflects in response to a force. Alternatively, a deflectable ferrite core can be used within an inductive coil may be used or a or a fiber Bragg grating formed within an optical fiber can be used, for example. Other axial force sensor units may be used to sense a resilient axial displacement of the shaft 3410 (e.g., relative to the proximally mounted mechanical structure, not shown). An axial force imparted to the end effector 3460 can cause axial displacement of the shaft 3410 in a direction along a center axis of the shaft (substantially parallel to the beam center axis). The axial force may be in the proximal direction (e.g., a reactive force resulting from pushing against tissue with the end effector) or it may be in the distal direction (e.g., a reactive force resulting from pulling tissue grasped with the end effector).

In some embodiments, X and/or Y forces imparted on the end effector 3460 may result in strain in the beam 3810 when the beam 3810 is deflected (e.g., displaced or bent). In other words, the X and/or Y forces deflect the center axis of the beam 3810 away from a designed position (similar to the deflection shown for the beam 2810 shown in FIG. 13) of the center axis, and, thus, relative to a center axis of the shaft 3410. Said another way, a distal end portion 3824 of the beam 3810 can bend relative to a proximal end portion 3822 of the beam 3810 such that the end portion 3824 of the beam 3810 is displaced a deflection distance relative to the designed position of the center axis.

As depicted, the outer shaft 3470 can limit the displacement of the beam 3810 and produce a reactive force that is exerted on the beam 3810. For example, the outer shaft 3470 can include or function as a hard stop (e.g., similar to the hard stop structure 2430 as depicted in FIGS. 11-14). In an embodiment wherein the displacement of beam 3810 is limited by the outer shaft 3470, alone or in combination with a shroud 3420 that surrounds a portion of the beam 3810, the strain distribution over the length of the beam 3810 may deviate relative to displacements of the beam 3810 that are not limited. Similarly stated, when the beam 3810 contacts the hard stop, the beam 3810 no longer behaves as a cantilevered beam. In this condition, the deflection is greater than the maximum deflection permitted at the location of the hard stop. As a result, the first strain sensor 3830 may produce signals that do not accurately represent the applied force affecting the end effector 3460. For example, when the applied force causes a deflection that results in the generation of the reactive force, the signals from the first strain sensor 3830 may indicate a force that is decreasing while the applied force acting on the end effector 3460 is actually increasing (e.g., a force inversion condition may exist). Insofar as the controller 1800 may utilize the signals from the first strain sensor 3830 to generate the haptic feedback delivered to the surgeon S, the inaccurate representation of the applied force resulting in inaccurate haptic feedback may be undesirable. It should therefore be appreciated that detecting such conditions and mitigating the impact of inaccurate haptic feedback may be beneficial to the operation of the surgical system 1000. It should be appreciated that in additional embodiments, the cannula structure 3600, the shroud 3420, and/or combinations thereof and also serve to limit the deflection of the beam 3810.

Accordingly, the controller 1800 can be configured to implement any of the methods and procedures described herein. Specifically, the controller can utilize the force sensor unit 3800 to detect the occurrence of the condition in which the deflection of the beam is greater than a deflection threshold and provide an indication of this condition (and, in some situations, take other actions). This is accomplished by receiving a second signal from the second strain sensor 3850 coupled to the outer shaft 3470 at a reference location RL (see e.g., the reference location RL as indicated in FIGS. 11-12). Based on the second signal, the magnitude of a reactive force resulting from contact between the beam 3810 and the outer shaft 3470 can be determined.

As depicted in FIG. 18, in some embodiments, the hard stop structure is the outer shaft 3470. The outer shaft 3470 includes a wall 3472. In some embodiments, the wall 3472 surrounds the beam 3810. In some embodiments, the wall 3472 surrounds a portion of the shroud 3420. In some embodiments, the wall 3472 surrounds a portion of the beam 3810 and a portion of the shroud 3420, which, in turn, also surrounds a portion of the beam 3810. Accordingly, in some embodiments, deflection of the beam 3810 in response to an applied force can be limited by contact with the wall 3472 of the outer shaft 3470 or a combination of the wall 3472 and an additional structure (e.g., the shroud 3420 and/or the cannula 3600). In some embodiments, the shroud 3420 can be configured as the outer shaft 3470.

In some embodiments, the wall 3472 includes an inner face 3474. The inner face 3474 is oriented toward the beam 3810. In some embodiments, the second strain sensor 3850 is coupled to the inner face 3474. As depicted, the second strain sensor 3850 can be coupled to the inner face 3474 at the reference location RL. Accordingly, the second strain sensor 3850 can be coupled to the inner face 3474 at the hard stop location. In other words, the second strain sensor 3850 is positioned such that the deflection of the beam 3810 brings the beam 3810 into compressive contact with the second strain sensors 3850.

Referring still to FIG. 18, in some embodiments, the second strain sensor 3850 includes at least one force sensor 3852. The force sensor(s) 3852 is coupled to a load ring 3854 that at least partially surrounds the beam 3810. In some embodiments, the load ring 3854 is a deformable member that has an annular structure. For example, the load ring 3854 can be an elastomeric member that surrounds the beam 3810 at the longitudinal position of the reference location RL. The force sensor(s) 3852 can be embedded in the elastomeric member. The force sensor(s) 3852 can, for example, be a load cell, a force transducer, a force sensing resistor, an optical force sensor, an ultrasonic force sensor, or a combination thereof. In other words, the force sensor(s) 3852 can be a sensing element configured to measure compressive loads exerted on the second strain sensor 3850 by the beam 3810.

As depicted in FIG. 18, in some embodiments, the second strain sensor 3850 includes more than one force sensor 3852. For example, in some embodiments, the second strain sensor 3850 includes two force sensors 3852 that are positioned to not be diametrically opposed. In some embodiments, the second strain sensor 3852 includes at least three force sensors 3852 that are distributed equidistantly about a cross-sectional circumference of the outer shaft 3470. In other words, the force sensors 3852 can be equally spaced along the inner face 3474 of the wall 3472 at the longitudinal position of the reference location RL. For example, the center of each of three force sensors 3852 can be separated by an arc of 120 degrees. Similarly, the center of each of four force sensors 3852 can be separated by an arc of 90 degrees.

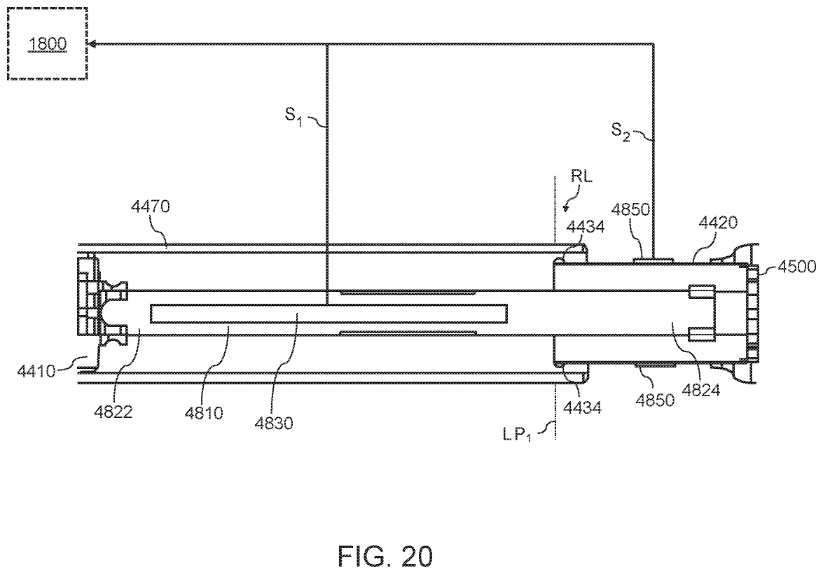

FIGS. 19 and 20 depict a perspective view and a cross-sectional side view taken at P-P of a medical instrument 4400 and a cannula 4600. In some embodiments, the instrument 4400 or any of the components thereof are optionally parts of a surgical system that performs surgical procedures. The surgical system may include a manipulator unit, a series of kinematic linkages, a series of cannulas, or the like. The instrument 4400 (and any of the instruments described herein) can be used in any suitable surgical system, such as the MIRS system 1000 shown and described above.

The instrument 4400 can include any of the elements and/or structures described herein with reference to instrument 3400 and perform any of the methods, procedures, and/or operations described herein. For example, in some embodiments, the instrument 4400 includes a proximal mechanical structure (not shown), an outer shaft 4470, a shroud 4420, a shaft 4410, a force sensor unit 4800, a wrist assembly 4500, and an end effector 4460, which can include at least one tool member 4462. The force sensor unit 4800 includes a beam 4810, a first strain sensor 4830 coupled to the beam 4810, and a second strain sensor 4850 coupled to the shroud 4420. As depicted, the shroud 4420 can be a hard stop structure as described herein. As depicted, in an embodiment, a shroud 4420 has a generally cylindrical shape that surrounds at least a portion of the beam 4810. In some embodiments, the shroud 4420 extends proximally from the wrist assembly 4500. However, in some embodiments, the shroud 4420 extends distally from the shaft 4410.

In some embodiments, the first strain sensor 4830 can, for example, include any of the elements disclosed herein with reference to the first strain sensor 2830, 3830 and may be used to measure forces imparted on the surgical instrument during a surgical procedure as described in more detail herein. In some embodiments, the beam 4810 can define at least three side surfaces disposed acutely to each other. In additional embodiments, the beam 4810 can define at least four side surfaces disposed perpendicular to one another. The first strain sensor 4830 may be mounted to the side surfaces in appropriate locations. The beam 4810 defines a beam center axis (see e.g., beam center axis A_{B} described above with reference to FIGS. 11-14) which can be aligned within a center axis (not shown) of the instrument shaft 4410. The beam center axis is a neutral axis that is equidistant from the sides (e.g., faces) of the beam 4810.

In use, the end effector 4460 may contact anatomical tissue, which may result in X, Y, or Z direction forces (similar to the forces exerted on the end effector 2460 shown in FIG. 13) being imparted on the end effector 4460. This contact may also result in forces about the various axes. The first strain sensor 4830 may be used to measure strain in the beam 4810 as a result of such forces imparted on the end effector 4460. More specifically, the first strain sensor 4830 can measure forces imparted on the end effector 4460 that are transverse (e.g., perpendicular) to a center axis of the beam 4810 as such forces are transferred to the beam 4810 in the X and Y directions (see FIG. 13). Specifically, the transverse forces acting upon the end effector 4460 can cause a slight bending of the beam 4810, which can result in a tensile strain imparted to one side of the beam 4810 and a compression strain imparted to an opposing side of the beam 4810. The first strain sensor 4830 may be coupled to the beam 4810 to measure such tensile and compression forces, with the resultant measurements being communicated to the controller via a communication coupling therebetween.

More specifically, when a force is imparted on a distal portion of the instrument 4400 (e.g., at end effector 4460) in the X or Y directions (see FIG. 11B for reference to X, Y and Z directions), such transverse force can cause the beam 4810 to bend (about either or some combination of the X axis or the Y axis), which can result in a tensile strain imparted to one side of the beam 4810 and a compression strain imparted to the opposite side of the beam 4810. The first strain sensor 4830 on the beam 4810 can measure such tensile and compression strains.

Although shown as including only the force sensor unit 4800, in some embodiments, the instrument 4400 (or any of the instruments described herein) can include additional force sensor units to measure the axial force(s) (i.e., in the direction of the Z-axis parallel to the beam center axis A_{B}) imparted on the end effector 4460. An axial force sensor unit in an example surgical instrument can comprise a deflectable planar diaphragm sensor that deflects in response to a force. Alternatively, a deflectable ferrite core can be used within an inductive coil may be used or a or a fiber Bragg grating formed within an optical fiber can be used, for example. Other axial force sensor units may be used to sense a resilient axial displacement of the shaft 4410 (e.g., relative to the proximally mounted mechanical structure, not shown). An axial force imparted to the end effector 4460 can cause axial displacement of the shaft 4410 in a direction along a center axis of the shaft (substantially parallel to the beam center axis). The axial force may be in the proximal direction (e.g., a reactive force resulting from pushing against tissue with the end effector) or it may be in the distal direction (e.g., a reactive force resulting from pulling tissue grasped with the end effector).

In some embodiments, X and/or Y forces imparted on the end effector 4460 may result in strain in the beam 4810 when the beam 4810 is deflected (e.g., displaced or bent). In other words, the X and/or Y forces deflect the center axis of the beam 4810 away from a designed position (similar to the deflection shown for the beam 2810 shown in FIG. 13) of the center axis, and, thus, relative to a center axis of the shaft 4410. Said another way, a distal end portion 4824 of the beam 4810 can bend relative to a proximal end portion 4822 of the beam 4810 such that the end portion 4824 of the beam 4810 is displaced a deflection distance relative to the designed position of the center axis.

In some embodiments, contact between the shroud 4420 and the beam 4810 or another structure, such as the outer shaft 4470 or the cannula 4600, can limit the displacement of the beam 4810 and produce a reactive force that is exerted on the beam 4810. For example, the shroud 4420 can include or function as a hard stop (e.g., similar to the hard stop structure 2430 as depicted in FIGS. 11-14). In an embodiment wherein the displacement of beam 4810 is limited by the shroud 4420 the strain distribution over the length of the beam 4810 may deviate relative to displacements of the beam 4810 that are not limited. Similarly stated, when the beam 4810 contacts the hard stop or the shroud 4420 contacts the outer shaft 4470, with sufficient force, the beam 4810 no longer behaves as a cantilevered beam. In this condition, the deflection is greater than the maximum deflection permitted at the location of the hard stop. As a result, the first strain sensor 4830 may produce a first signal S₁ that does not accurately represent the applied force affecting the end effector 4460. For example, when the applied force causes a deflection that results in the generation of the reactive force, the first signal S₁ from the first strain sensor 4830 may indicate a force that is decreasing while the applied force acting on the end effector 4460 is actually increasing (e.g., a force inversion condition may exist). Insofar as the controller 1800 may utilize the first signal S₁ to generate the haptic feedback delivered to the surgeon S, the inaccurate representation of the applied force resulting in inaccurate haptic feedback may be undesirable. It should therefore be appreciated that detecting such conditions and mitigating the impact of inaccurate haptic feedback may be beneficial to the operation of the surgical system 1000.

Accordingly, the controller 1800 can be configured to implement any of the methods and procedures described herein. Specifically, the controller can utilize the force sensor unit 4800 to detect the occurrence of the condition in which the deflection of the beam is greater than a deflection threshold and provide an indication of this condition (and, in some situations, take other actions). This is accomplished by receiving a second signal S₂ from the second strain sensor 4850 coupled to the shroud 4420. Based on the second signal S₂, the magnitude of a reactive force resulting from contact between the shroud 4420 and the beam 4810 or the outer shaft 4470 can be determined.

As depicted in FIG. 19, in some embodiments, the hard stop structure (as described herein with reference to hard stop structure 2430) is the shroud 4420. The shroud 4420 at least partially surrounds the beam 4810. In some embodiments, the shroud 4420 is at least partially surrounded by the outer shaft 4470 and/or the cannula 4600. For example, in some embodiments, the shroud 4420 includes a wall 4422 that surrounds the distal end portion 4824 of the beam 4810. The shroud 4420 defines a hard stop engagement locator 4434 that is configured to engage the outer shaft 4470 or the beam 4810 in response to the deflection of the beam 4810. The hard stop engagement locator 4434 can, for example, be a protrusion that is integral to the shroud 4420 or coupled thereto. The hard stop engagement locator 4434 defines a longitudinal position LP₁ of the reference location RL. In other words, the hard stop engagement locator 4434 establishes the location at which the outer shaft 4470 or the beam 4810 are contacted by the shroud 4420 in response to the deflection. The hard stop engagement locator 4434 ensures that the contact location between the shroud 4420 and the beam 4810 or the outer shaft 4470 is at a fixed position. For example, the hard stop engagement locator 4434 ensures that the contact location between the shroud 4420 and the outer shaft 4470 is unchanged even when there's relative movement between the shroud 4420 and the outer shaft 4470 even if there is relative movement between the shroud 4420 and the outer shaft 4470.

In some embodiments, the second strain sensor 4850 includes one or more strain gauges that are coupled to the shroud 4420. The strain gauge(s) generates an output voltage (e.g. the second signal S₂) that is indicative of a strain of the wall 4422. In some embodiments, at least three strain gauges are distributed equally about the circumference of the shroud at a single longitudinal position. For example, the center of each of three strain gauges can be separated by an arc of 120 degrees. Similarly, the center of each of the strain gauges can be separated by an arc of 90 degrees. In an embodiment wherein the shroud 4420 extends proximally from the wrist assembly 4500, the strain gauge(s) is positioned between the wrist assembly 4500 and the hard stop engagement locator 4434. In an embodiment wherein the shroud 4420 extends distally from the shaft 4410, the strain gauge(s) is positioned between the shaft 4410 and the hard stop engagement locator 4434.

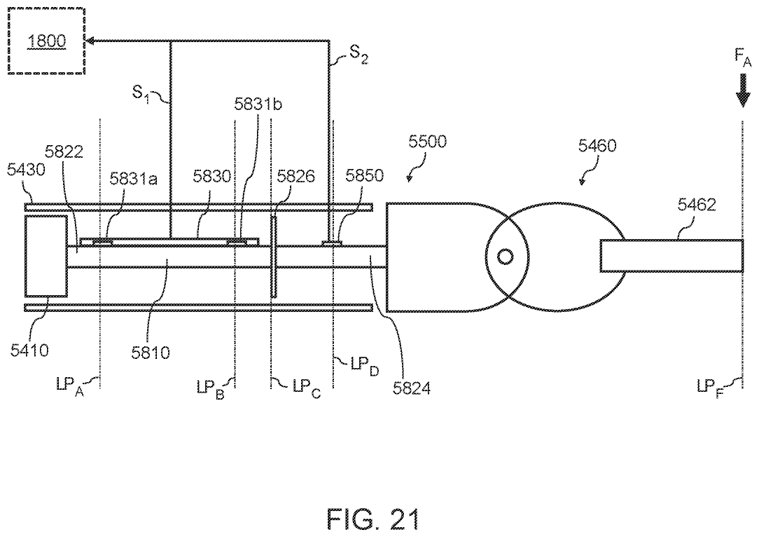

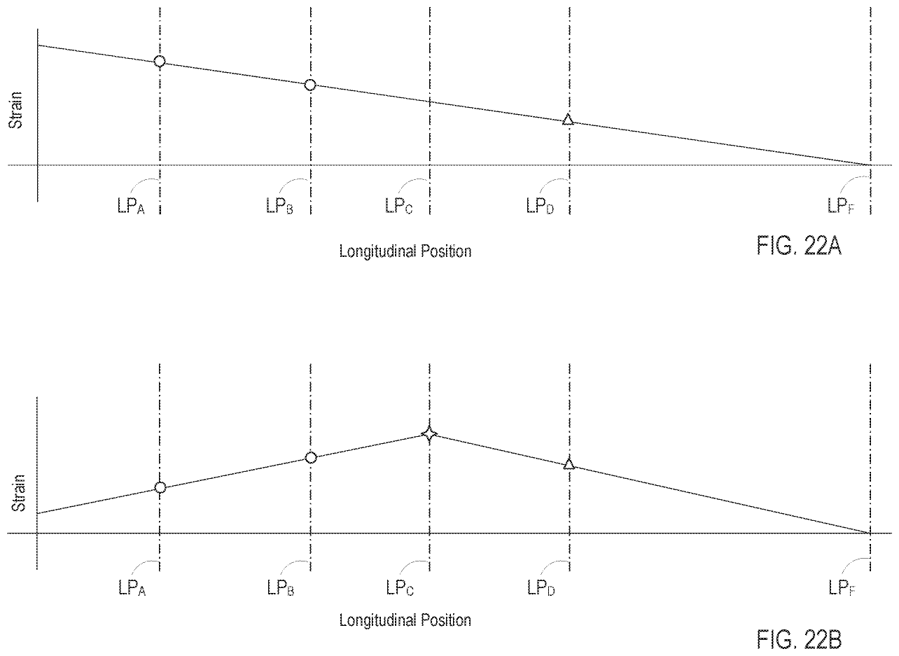

FIG. 21 is a diagrammatic illustration of a portion of a medical instrument 5400 according to an embodiment. FIG. 22A is a graph showing strain (Y-axis) as a function of the longitudinal positions (X-axis) along the beam 5810 during operation of the instrument 5400 on a condition in which the beam deflection is less than a deflection threshold, while FIG. 22B is a graph showing strain (Y-axis) as a function of the longitudinal positions (X-axis) on a condition in which the beam deflection is greater than the deflection threshold. In some embodiments, the instrument 5400 or any of the components thereof are optionally parts of a surgical system that performs surgical procedures. The surgical system may include a manipulator unit, a series of kinematic linkages, a series of cannulas, or the like. The instrument 5400 (and any of the instruments described herein) can be used in any suitable surgical system, such as the MIRS system 1000 shown and described above.

The instrument 5400 can include any of the elements and/or structures described herein with reference to instrument 2400, instrument 3400 or instrument 4400 and perform any of the methods, procedures, and/or operations described herein. For example, in some embodiments, the instrument 5400 includes a hard stop structure 5430, a shaft 5410, a force sensor unit 5800, a wrist assembly 5500, and an end effector 5460, which can include at least one tool member 5462. The force sensor unit 5800 includes a beam 5810, a contact feature 5826, a first strain sensor 5830, and a second strain sensor 5850. The beam 5810 is surrounded, at least partially, by the hard stop structure 5430. The contact feature 5826 is coupled to the beam 5810 at a longitudinal location LP_{C} that corresponds to the reference location RL. In other words, the contact feature 5826 establishes the hard stop location in at a fixed, known longitudinal position at which deflection of the beam 2810 in response to an applied force F_{A} is limited by contact between the contact feature 5826 and the hard stop structure 5430. Accordingly, in some embodiments, contact between the contact feature 5826 and the reference location RL equates to contact between the distal end portion 5824 of the beam and the reference location RL.

In some embodiments, the first strain sensor 5830 is coupled to the beam 5810 at a longitudinal position that is proximal to the contact feature 5826. In other words, the first strain sensor 5830 is positioned longitudinally between the reference location RL and the proximal end portion 5822 of the beam 5810. The second strain sensor 5850 is coupled to the beam at a longitudinal position that is distal to the contact feature 5826. In other words, the second strain sensor 5850 is positioned longitudinally between the reference location RL and the distal end portion 5824 of the beam 5810.

In some embodiments, the first strain sensor 5830 can, for example, include any of the elements disclosed herein with reference to the first strain sensor 2830, 3830, 4830 and can be used to measure forces imparted on the surgical instrument during a surgical procedure as described in more detail herein. In some embodiments, the beam 5810 can define at least three side surfaces disposed acutely to each other. In additional embodiments, the beam 5810 can define at least four side surfaces disposed perpendicular to one another. The first strain sensor 5830 can be mounted to the side surfaces in appropriate locations. The beam 5810 defines a beam center axis (see e.g., beam center axis A_{B} described above with reference to FIGS. 11-14) which can be aligned within a center axis (not shown) of the instrument shaft 5410. The beam center axis is a neutral axis that is equidistant from the sides (e.g., faces) of the beam 5810.

As depicted in FIG. 21, in some embodiments, the first strain sensor 5830 includes at least two electrical strain sensing circuits (e.g., bridge circuits), and other strain sensor configurations are contemplated (e.g., optical fiber Bragg grating sensors, piezoelectric sensors, and the like). For example, the first strain sensor 5830 can include a first half-bridge circuit 5831a and a second half-bridge circuit 5831b positioned at different longitudinal positions along the beam 5810. As described herein, each bridge circuit (and also each strain sensor) includes one or more strain gauges (e.g., tension strain gauge resistor(s) or compression strain gauge resistor(s)). As depicted, the first half-bridge circuit 5831a can be coupled to the beam 5810 at longitudinal position A (LP_{A}). The second half-bridge circuit 5831b is coupled to the beam 5810 at longitudinal position B (LP_{B}) that is between the first half-bridge circuit 5831a and the contact feature 5826. The first strain sensor 5830 generates the first signal S₁ in response to the applied force F_{A} at longitudinal position F (LP_{F}) that is received by the controller 1800.

In some embodiments second strain sensor 5850 can include at least one strain gauge resistor and/or at least one bridge circuit. The second strain sensor 5850 is coupled to the beam at longitudinal position D (LP_{D}). The second strain sensor 5830 generates the second signal S₂ in response to the applied force F_{A} at longitudinal position F (LP_{F}) that is received by the controller 1800.

In some embodiments, the controller 1800 is configured to determine a proximal strain function for the first signal S₁. For example, as depicted in FIGS. 22A and 22B, the proximal strain function can be a linear function. The slope of the linear function is determined by the output of the first half-bridge circuit 5831a at longitudinal position A (LP_{A}) and the output of the second half-bridge circuit 5831b at longitudinal position B (LP_{B}) in response to the force applied at longitudinal position F (LP_{F}). Insofar as the beam 5810 is a cantilevered structure, the slope of the proximal strain function is negative when the beam deflection is less than the deflection threshold as depicted in FIG. 22A. In other words, in the absence of significant contact between the contact feature 5826 and the hard stop structure 5430, the first half-bridge circuit 5831a at longitudinal position A (LP_{A}) generates a maximal strain measurement. In accordance with the proximal strain function, the magnitude of the measured strain then decreases linearly to longitudinal position F (LP_{F}) (e.g., the point of application of the applied force F_{A}). When the beam deflection is less than the deflection threshold, the second signal S₂ (e.g., the output of the second strain sensor 5850 at longitudinal position D (LP_{D})) conforms to the proximal strain function.

As depicted in FIG. 22B, the second signal S₂ is indicative of the contact feature 5826 being in contact with the hard stop structure 5430 (i.e., contact between the distal end portion 5824 of the beam 5810 and the reference location RL) when the second signal S₂ deviates from the proximal strain function of the first signal S₁. For example, in the presence of significant contact between the contact feature 5826 and the hard stop structure 5430, the proximal strain function can be a linear function having a positive slope. As depicted, the slope can indicate an increasing strain magnitude from longitudinal position A (LP_{A}) to the longitudinal location LP_{C} of the contact feature 5826 and the reference location RL. As the magnitude of the strain indicated by the second signal S₂ is less than a predicted magnitude at longitudinal position D (LP_{D}) based on the proximal strain function, the second signal S₂ deviates from the proximal strain function. This deviation is indicative of deflection of the beam 5810 being limited by the hard stop structure 5430. Said another way, this deviation indicates that the deflection of the beam is larger than the deflection threshold.

Referring still to FIG. 22B, in some embodiments, the controller 1800 can be configured to determine the magnitude of the applied force F_{A} based on the strain functions of the first signal S₁ and the second signal S₂. For example, the line generated by the proximal strain function can be extended to the known point of contact (i.e. longitudinal location LP_{C}) with the hard stop structure 5430 established by contact feature 5826. Thus, the strain magnitude at the longitudinal location LP_{C} of the contact feature 5826 (e.g., at the reference location RL) can be determined based on the proximal strain function. This strain magnitude can be utilized with the strain magnitude indicated by the second signal S₂ at longitudinal position D (LP_{D}) to determine a distal strain function.

As depicted in FIG. 22B, the distal strain function can have a negative slope. In accordance with the distal strain function, the magnitude of the measured strain then decreases linearly from longitudinal location LP_{C} to longitudinal position F (LP_{F}) (e.g., the point of application of the applied force F_{A}). The slope of the distal strain function represents the strain in the portion of the beam that is distal to the contact feature 5826 and, thus, is proportional to the applied force F_{A}. Said another way, in that the contact between the contact feature of 826 and the hard stop structure 5430 establishes a mechanical ground between the beam 5810 and the hard stop structure 5430, the portion of the beam 5810 that is distal to the contact feature is cantilevered. The strain in the cantilevered distal portion of the beam 5810 is described by the distal strain function. It should be appreciated that determining the magnitude of the applied force F_{A} based on the distal strain slope is associated with the reactive force in that, in accordance with Eq. 3, the determined force F (as indicated by the first signal S₁ from the first strain sensor 5830) corresponds to the applied force F_{A} minus the reactive force.

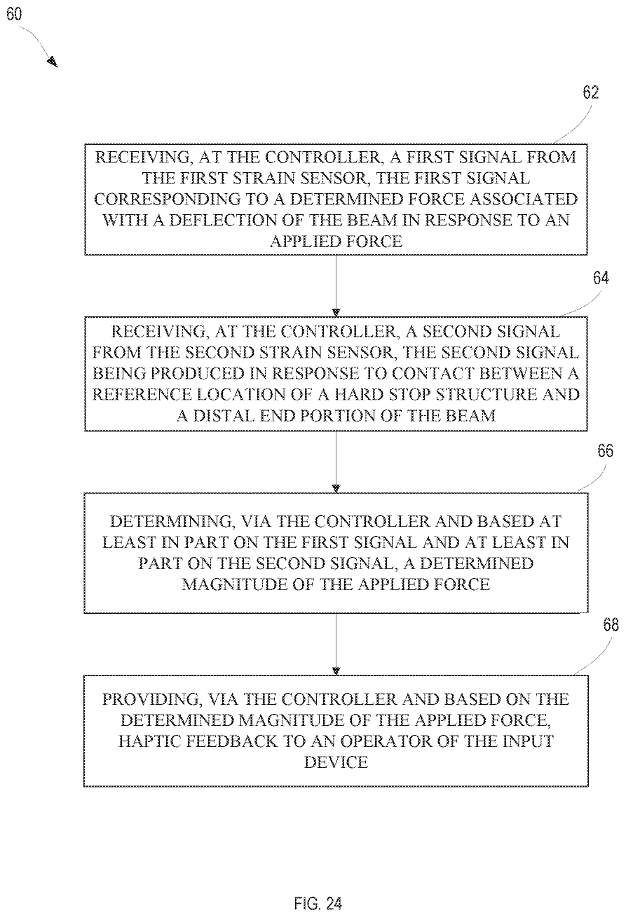

FIG. 24 is a flow chart of a method 60 of control for a surgical system according to an embodiment. The method 60 may, in an embodiment, be performed via a teleoperated system, such as system 1000 as described with reference to FIGS. 4-23. However, it should be appreciated that in various embodiments, aspects of the method 60 can be accomplished via additional embodiments of the system 1000 or components thereof, such as instrument 2400, instrument 3400, instrument 4400 and or instrument 5400 as described herein. Accordingly, the method 60 can be implemented on any suitable device as described herein. Thus, it should be understood that the method 60 can be employed using any of the medical devices/instruments and controllers described herein.

As depicted at 62 in FIG. 24, the controller receives a first signal from the first strain sensor. The first signal corresponds to a determined force that is associated with a deflection of the beam in response to an applied force. As depicted at 64, the controller receives a second signal from the second strain sensor. The second signal is produced in response to contact between a reference location of a hard stop structure and a distal end portion of the beam. In some embodiments, the distal end portion of the beam can include a contact feature (e.g., contact feature 5826 described herein). As such, in some embodiments, the second signal is indicative of whether the distal end portion of the beam is in contact with the hard stop structure at the reference location. For example, in some embodiments, the second signal corresponds to a reactive force magnitude that is greater than a reactive-force minimum threshold when the distal end portion of the beam is in contact with the hard stop structure. By way of illustration, in some embodiments, the reactive-force minimum threshold can be established at a magnitude of zero.

In some embodiments, as depicted at 66, the magnitude of the applied force is determined by the controller based at least in part on the first signal and at least in part on the second signal in accordance with method 60. As depicted at 68, haptic feedback is provided to the control unit of the surgical system (e.g., to an operator of the input device) via the controller based on the determined magnitude of the applied force. On a condition in which the distal end portion of the beam is not in contact with the hard stop at the reference location, the haptic feedback is a designed haptic feedback. However, on a condition in which the distal end portion of the beam is in contact with the hard stop at the reference location, the haptic feedback can be a modified haptic feedback. In some embodiments, the controller can provide an indication to the operator of the input device that the modified haptic feedback is provided to, or is available to be provided to, the input device. Said another way, on a first condition in which the deflection is greater than the deflection threshold the controller can provides the indication to the operator of the surgical system (e.g., the surgical system 1000) that the modification of the haptic feedback is provided to, or is available to, be provided to the control unit (e.g., control unit 1100).

In some embodiments, the modification of the haptic feedback corresponds to a full restriction of a designed haptic feedback. In such embodiments, the magnitudes of the modified haptic feedback along each axis are each less than corresponding designed haptic feedback magnitudes. However, in some embodiments, the modified haptic feedback corresponds to a partial restriction of the designed haptic feedback. For example, in such embodiments, the magnitudes of the modified haptic feedback along one axis may be less than the corresponding designed haptic feedback magnitude while magnitudes along the other axes are unaffected.

In some embodiments, the second signal corresponds to a reactive force magnitude that is greater than a reactive-force maximal threshold. In such an embodiment, the method includes halting an operation of the surgical system and the modified haptic feedback corresponds to the halted state of the surgical system. For example, when the reactive force magnitude indicated by the second signal is greater than the reactive-force maximal threshold, the controller can halt a movement of the instrument such that the end effector is maintained in a fixed position until the reactive force magnitude is less than the reactive-force maximal threshold.

As described herein, in some embodiments, the force sensor unit includes a contact feature (e.g., contact feature 5826) positioned at a longitudinal location of the beam corresponding to the reference location. The contact feature, being an element of the distal end portion of the beam, fixes longitudinally the point at which the distal end portion of the beam contacts the reference location. In such embodiments, the first strain sensor is positioned longitudinally between the reference location and a proximal end portion of the beam, while the second strain sensor is on the beam and positioned longitudinally between the reference location and the distal end portion of the beam. Based on the longitudinal positioning of the elements of the first strain sensor, the controller determines a linear, proximal strain function. The proximal strain function can generate a line that extends in a distal direction from the first strain sensor, through the longitudinal location of the contact feature, through the longitudinal position of the second strain sensor, and to a resolved point of application of an applied force. On a condition in which the second signal deviates from the proximal strain function (e.g., a plot of the strain magnitude at the longitudinal position of the second strain sensor does not fall on the line generated by the proximal strain function), the second signal is indicative of contact between the distal end portion of the beam and the hard stop structure (e.g., hard stop structure 2430, 5430).

In some embodiments wherein the force sensor unit includes the contact feature, the controller can determine the magnitude of the applied force based in part, on the proximal strain function. For example, the controller determines a strain magnitude at the longitudinal location of the beam corresponding to the reference location based on the proximal strain function derived from first strain signal. Said another way, the strain magnitude at the longitudinal location can be determined based on the line generated by the proximal strain function that extends through the longitudinal location of the contact feature. The controller can then determine a distal strain function based on the strain magnitude at the reference location and the second strain signal. In so far as the contact between the contact feature and the hard stop structure establishes a mechanical ground between the beam and the hard stop structure, the portion of the beam that is distal to the contact feature is cantilevered. The strain in the cantilevered distal portion of the beam is described by the distal strain function. As such, the determined magnitude of the applied force is proportional to the slope of the distal strain function.

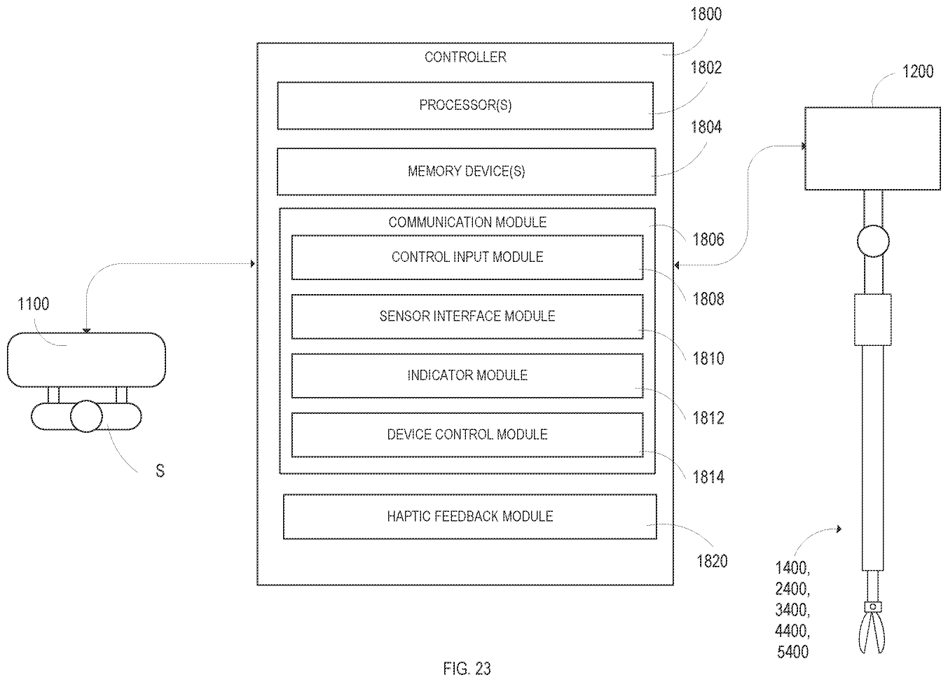

As shown particularly in FIG. 23, a schematic diagram of one embodiment of suitable components that may be included within the controller 1800 is illustrated. In some embodiments, the controller 1800 is positioned within a component of the surgical system 1000, such as the user control unit 1100 and/or the optional auxiliary equipment unit 1150. However, the controller 1800 may also include distributed computing systems wherein at least one aspect of the controller 1800 is at a location which differs from the remaining components of the surgical system 1000 for example, at least a portion of the controller 1800 may be an online controller.

As depicted, the controller 1800 includes one or more processor(s) 1802 and associated memory device(s) 1804 configured to perform a variety of computer implemented functions (e.g., performing the methods, steps, calculations and the like and storing relevant data as disclosed herein). Additionally, in some embodiments, the controller 1800 includes a communication module 1806 to facilitate communications between the controller 1800 and the various components of the surgical system 1000.

As used herein, the term “processor” refers not only to integrated circuits referred to in the art as being included in a computer, but also refers to a controller, a microcontroller, a microcomputer, a programmable logic controller (PLC), an application specific integrated circuit, and other programmable circuits. Additionally, the memory device(s) 1804 may generally comprise memory element(s) including, but not limited to, computer readable medium (e.g., random access memory (RAM)), computer readable nonvolatile medium (e.g., a flash memory), a floppy disk, a compact disc read only memory (CD ROM), a magneto optical disk (MOD), a digital versatile disc (DVD) and/or other suitable memory elements. Such memory device(s) 1804 may generally be configured to store suitable computer readable instructions that, when implemented by the processor(s) 1802, configure the controller 1800 to perform various functions.

In some embodiments, the controller 1800 includes a haptic feedback module 1820. The haptic feedback module 1820 may be configured to deliver a haptic feedback to the operator S based on inputs received from a force sensor unit of the instrument 1400 (e.g., the force sensor unit 2800, including the first strain sensor 2830 and the second strain sensor 2850 (FIG. 11). In some embodiments, haptic feedback module 1820 may be an independent module of the controller 1800. However, in some embodiments the haptic feedback module 1820 may be included within the memory device(s) 1804.

The communication module 1806 may include a control input module 1808 configured to receive control inputs from the operator/surgeon S, such as via the input device 1116 of the user control unit 1100. The communication module may also include an indicator module 1812 configured to generate various indications in order to alert the operator S.

The communication module 1806 may also include a sensor interface 1810 (e.g., one or more analog to digital converters) to permit signals transmitted from one or more sensors (e.g., the first strain sensors 2830 and the second strain sensor 2850 of the force sensor unit 2800 (FIG. 11)) to be converted into signals that can be understood and processed by the processors 1802. The sensors may be communicatively coupled to the communication module 1806 using any suitable means. For example the sensors may be coupled to the communication module 1806 via a wired connection and/or via a wireless connection, such as by using any suitable wireless communications protocol known in the art. Additionally, in some embodiments, the communication module 1806 includes a device control module 1814 configured to modify an operating state of the instrument 1400 (and/or any of the instruments described herein (e.g., 2400, 3400, 4400, 5400). Accordingly, the communication module is communicatively coupled to the manipulator 1200 and/or the instrument 1400. For example, the communications module 1806 may communicate to the manipulator 1200 and/or the instrument 1400 an excitation voltage for the strain sensor(s), a handshake and/or excitation voltage for a positional sensor (e.g., for detecting the position of the designated portion relative to the cannula), cautery controls, positional setpoints, and/or an end effector operational setpoint (e.g., gripping, cutting, and/or other similar operation performed by the end effector).

While various embodiments have been described above, it should be understood that they have been presented by way of example only, and not limitation. Where methods and/or schematics described above indicate certain events and/or flow patterns occurring in certain order, the ordering of certain events and/or operations may be modified. While the embodiments have been particularly shown and described, it will be understood that various changes in form and details may be made.

For example, any of the instruments described herein (and the components therein) are optionally parts of a surgical assembly that performs minimally invasive surgical procedures, and which can include a manipulator unit, a series of kinematic linkages, a series of cannulas, or the like. Thus, any of the instruments described herein can be used in any suitable surgical system, such as the MIRS system 1000 shown and described above. Moreover, any of the instruments shown and described herein can be used to manipulate target tissue during a surgical procedure. Such target tissue can be cancer cells, tumor cells, lesions, vascular occlusions, thrombosis, calculi, uterine fibroids, bone metastases, adenomyosis, or any other bodily tissue. The presented examples of target tissue are not an exhaustive list. Moreover, a target structure can also include an artificial substance (or non-tissue) within or associated with a body, such as for example, a stent, a portion of an artificial tube, a fastener within the body or the like.

For example, any of the components of a surgical instrument as described herein can be constructed from any material, such as medical grade stainless steel, nickel alloys, titanium alloys or the like. Further, any of the links, tool members, beams, shafts, cables, or other components described herein can be constructed from multiple pieces that are later joined together. For example, in some embodiments, a link can be constructed by joining together separately constructed components. In other embodiments however, any of the links, tool members, beams, shafts, cables, or components described herein can be monolithically constructed.

Although various embodiments have been described as having particular features and/or combinations of components, other embodiments are possible having a combination of any features and/or components from any of embodiments as discussed above. Aspects have been described in the general context of medical devices, and more specifically surgical instruments, but inventive aspects are not necessarily limited to use in medical devices.

What is claimed is:

1. A force sensor unit, comprising:
a beam at least partially surrounded by a hard stop structure;
a first strain sensor coupled to the beam; and
a second strain sensor;
wherein the beam includes a proximal end portion and a distal end portion;
wherein the hard stop structure includes a reference location at which the distal end portion of the beam contacts the hard stop structure on a condition in which a deflection of the beam is larger than a beam deflection threshold;
wherein the first strain sensor generates a first signal associated with the deflection of the beam; and
wherein the second strain sensor produces a second signal in response to contact between the reference location and the distal end portion of the beam.

2. The force sensor unit of claim 1, wherein:
the hard stop structure is any of a shroud or an outer shaft and surrounds at least a portion of the beam; and
the second strain sensor is positioned on the hard stop structure.

3. The force sensor unit of claim 2, wherein:
the hard stop structure is the shroud;
the shroud includes a wall surrounding the distal end portion of the beam;
the shroud defines a hard stop engagement locator configured to engage the outer shaft or the beam in response to the deflection of the beam; and
the hard stop engagement locator defines a longitudinal position of the reference location.

4. The force sensor unit of claim 3, wherein:
the second strain sensor includes one or more strain gauges coupled to the shroud; and
the one or more strain gauges generate an output voltage indicative of a strain of the wall.

5. The force sensor unit of claim 2, wherein:
the hard stop structure is the outer shaft;
the outer shaft further includes a wall surrounding any of the beam and a portion of the shroud;
the wall includes an inner face; and
the second strain sensor is coupled to the inner face of the wall at the reference location.

6. The force sensor unit of claim 5, wherein:
the second strain sensor includes at least one force sensor operably coupled to a load ring; and
the load ring is a deformable member.

7. The force sensor unit of claim 6, wherein:
the deformable member is an elastomeric member;
the second strain sensor includes three or more force sensors embedded in the elastomeric member; and
the three or more force sensors are distributed equidistantly about a cross-sectional circumference of the outer shaft.

8. The force sensor unit of claim 1, wherein:
the force sensor unit includes a contact feature positioned at a longitudinal location of the beam corresponding to the reference location;
the second strain sensor is on the beam;
the first strain sensor is positioned longitudinally between the reference location and the proximal end portion of the beam; and
the second strain sensor is positioned longitudinally between the reference location and the distal end portion of the beam.

9. The force sensor unit of claim 8, wherein:
the force sensor unit is coupled between a surgical instrument shaft and a surgical instrument wrist assembly of a surgical instrument;
the proximal end portion of the beam is coupled to the surgical instrument shaft; and
the distal end portion of the beam is coupled to the surgical instrument wrist assembly.

10. A surgical system, comprising:
a manipulator unit;
a medical instrument supported by and operated by the manipulator unit and including a force sensor unit;
an input device; and
a controller operably coupling the input device and the manipulator unit, operatively coupling the force sensor unit and the input device, and including a logic system and a memory system;
wherein the force sensor unit includes a beam, a first strain sensor, and a second strain sensor;
wherein the beam includes a proximal end portion and a distal end portion;
wherein the first strain sensor is positioned to produce a first signal associated with a deflection of the beam;
wherein a hard stop structure at least partially surrounds the beam;
wherein the hard stop structure includes a reference location at which the distal end portion of the beam contacts the hard stop structure on a condition in which a deflection of the beam is larger than a beam deflection threshold; and
wherein the controller includes a haptic feedback module, and the controller is configured to perform a plurality of operations including:
receiving a first signal from the first strain sensor, the first signal being associated with a strain associated with the deflection of the beam in response to an applied force,
receiving a second signal from the second strain sensor, the second signal being produced in response to contact at the distal end portion of the beam and the reference location,
determining, via the controller and based at least in part on the first signal and at least in part on the second signal, a determined magnitude of the applied force, and
providing, via the haptic feedback module and based on the determined magnitude of the applied force, haptic feedback to a human operator of the input device.

11. The surgical system of claim 10, wherein:
the second signal is indicative of whether the distal end portion of the beam is in contact with the hard stop structure at the reference location;
on a condition in which the distal end portion of the beam is not in contact with the hard stop at the reference location, the haptic feedback is a designed haptic feedback; and
on a condition in which the distal end portion of the beam is in contact with the hard stop at the reference location, the haptic feedback is a modified haptic feedback.

12. The surgical system of claim 11, further comprising:
providing, via the controller, an indication to the operator of the input device that the modified haptic feedback is provided to, or is available to be provided to, the input device.

13. The surgical system of claim 11, wherein:
the force sensor unit includes a contact feature positioned at a longitudinal location of the beam corresponding to the reference location;
the second strain sensor is on the beam;
the first strain sensor is positioned longitudinally between the reference location and the proximal end portion of the beam;
the second strain sensor is positioned longitudinally between the reference location and the distal end portion of the beam; and
the second signal is indicative of the distal end portion of the beam being in contact with the hard stop structure at the reference location on a condition when the second signal deviates from a proximal strain function of the first signal.

14. The surgical system of claim 13, wherein:
determining the magnitude of the applied force includes:
determining, via the controller, a strain magnitude at the longitudinal location of the beam corresponding to the reference location based on the proximal strain function, and
determining, via the controller, a distal strain function based on the strain magnitude at the longitudinal location and the second signal; and
the determined magnitude of the applied force is proportional to a slope of the distal strain function.

15. The surgical system of claim 11, wherein:
the second signal corresponds to a reactive force magnitude that is larger than a reactive-force minimum threshold.

16. The surgical system of claim 11, wherein:
the second signal corresponds to a reactive force magnitude that is larger than a reactive-force maximal threshold;
on a condition when the reactive force magnitude is larger than the reactive-force maximal threshold, the plurality of operations include halting an operation of the surgical system; and
the modified haptic feedback corresponds to the halted state of the surgical system.

17. The surgical system of claim 10, wherein:
the hard stop structure is any of a shroud or an outer shaft and surrounds at least a portion of the beam; and
the second strain sensor is positioned on the hard stop structure.

18. The surgical system of claim 17, wherein:
the hard stop structure is the shroud;
the shroud includes a wall surrounding the distal end portion of the beam;
the shroud defines a hard stop engagement locator configured to engage the outer shaft or the beam in response to the deflection of the beam; and
the hard stop engagement locator defines a longitudinal position of the reference location.

19. The surgical system of claim 18, wherein:
the second strain sensor includes one or more strain gauges coupled to the shroud; and
the one or more strain gauges generate an output voltage indicative of a strain of the wall.

20. The surgical system of claim 17, wherein:
the hard stop structure is the outer shaft;
the outer shaft further includes a wall surrounding any of the beam and a portion of the shroud;
the wall includes an inner face; and
the second strain sensor is coupled to the inner face of the wall at the reference location.

21. The surgical system of claim 20, wherein:
the second strain sensor includes at least one force sensor operably coupled to a load ring; and
the load ring is a deformable member.

22. The surgical system of claim 21, wherein:
the deformable member is an elastomeric member;
the second strain sensor includes three or more force sensors embedded in the elastomeric member; and
the three or more force sensors are distributed equidistantly about a cross-sectional circumference of the outer shaft.

23. The surgical system of claim 10, wherein:
the surgical system includes a surgical instrument shaft and a surgical instrument wrist assembly;
the proximal end portion of the beam is coupled to the surgical instrument shaft; and
the distal end portion of the beam is coupled to the surgical instrument wrist assembly.
